(19) United States
(12) Patent Application Publication
(10) Pub. No.: US 2026/0286341 A1
(43) Pub. Date: Sep. 24, 2026

(54) METHOD OF IDENTIFYING, CHARACTERISING AND/OR DESIGNING AGENT OR A TARGET BINDING SITE OF AN AGENT

(71) Applicant: AAX BIOTECH AB, Stockholm (SE)

(72) Inventors: Daniel Gunnar JOHANSSON, Stockholm (SE); Mats Axel Atterdag PERSSON, Bromma (SE); Anders ANDERSSON, Solna (SE)

(21) Appl. No.: 19/474,869

(22) PCT Filed: Apr. 17, 2024
(86) PCT No.: PCT/EP2024/060425

§ 371 Date: Oct. 13, 2025

(30) Foreign Application Priority Data
Apr. 18, 2023 (GB) 2305642.7

(51) Int. Cl. C12N 15/10

(52) U.S. Cl. CPC C12N 15/1037

(57) ABSTRACT

The present invention relates to a method for identifying and/or characterising the target binding site of an agent or fragment thereof, a method for identifying a target binding site of an agent or fragment thereof and/or designing a target binding site to which binding of an agent or fragment thereof is increased or decreased, and a method for identifying an agent or fragment thereof and/or designing an agent or fragment thereof, to which binding of a target antigen or fragment thereof, is increased or decreased. The present invention also relates to a library of displayed polypeptides or a library of displayed agents obtainable by the methods disclosed herein.

The present invention relates to a method for identifying and/or characterising the target binding site of an agent or fragment thereof, a method for identifying a target binding site of an agent or fragment thereof and/or designing a target binding site to which binding of an agent or fragment thereof is increased or decreased, and a method for identifying an agent or fragment thereof and/or designing an agent or fragment thereof, to which binding of a target antigen or fragment thereof, is increased or decreased. The present invention also relates to a library of displayed polypeptides or a library of displayed agents obtainable by the methods disclosed herein.

Antibodies are able to selectively recognise and bind to a cognate antigen target, and that property has led to them being widely used in research, diagnostics and therapy applications. Other agents (e.g. small molecules and aptamers) also bind to specific regions of their target antigen, and therefore have similar utility. The area of the antigen that is bound by an antibody or other specific-targeting agent is known as the epitope, and so mapping the epitope recognised by such molecules can provide a valuable insight into the nature of the agent-antigen interaction and/or the mechanism of action of an antigen (e.g. why a virus can/cannot infect when bound by a particular antibody/agent). Epitope mapping is also used to assist lead candidate selection in therapeutic antibody development and small molecule screening since in some cases only binding of a specific epitope elicits the desired effect or has an influence on a pathology.

Many methods have been developed to define antibody/agent epitopes, which can be broadly grouped into either high-throughput or high-resolution methods. Methods with high resolution are often capable of identifying more complex epitopes such as discontinuous or structural epitopes that are dependent on correct antigen folding. In contrast, known high-throughput methods are typically only able to resolve linear epitopes.

In order to identify the epitope of an antigen recognised by a particular antibody/agent, mutational scanning of the antigen can be carried out. Mutational scanning approaches work on the assumption that introducing a mutation at an amino acid position in an antigen that forms part of the epitope will result in loss of or decreased binding of the antibody/agent to the antigen. Traditional mutational scanning (such as alanine scanning) is generally very labour intensive and requires the cloning, expression, and purification of hundreds of antigen mutants.

A general challenge with traditional mutational scanning is the difficulty in differentiating between an amino acid being part of the recognised epitope versus an amino acid that is critical for the overall structure of the antigen, since in both cases the tested agent may not be able to bind the antigen but for different reasons.

Structurally important amino acids are typically found inside the core of the protein and therefore have low solvent accessibility (SA) values. Accordingly, one way of filtering structurally important residues from actual epitope residues is to use an SA cut-off threshold. SA values can be predicted using a variety of algorithms, or they can be extracted from crystal structures. As such, the SA values do not necessarily reflect the protein structure in solution. In addition, an antibody/agent may in some cases alter the structure of its target upon binding thereby exposing amino acids previously buried in the antigen structure. Those factors further complicate the use of SA value as the sole means by which amino acids are filtered during epitope mapping.

Moreover, it can be very challenging for a user to decide what SA value cut-off to use in a given epitope mapping experiment (e.g. because the structural context and range of SA values changes from antigen to antigen). Accordingly, the choice of SA value cut-off is often arbitrary and can lead to inappropriate inclusion or exclusion of amino acids in the epitope mapping output. For instance, using too low of an SA value cut-off can result in structurally important residues being assigned as part of the epitope (i.e. false positives), whereas having too high of an SA value cut-off can inadvertently exclude residues that are actually part of the epitope (i.e. false negatives). Consequently, this can lead to misidentification of the epitope in an antigen that is recognised by an antibody/agent.

Thus, there exists a need for methods of identifying, characterising and/or designing agent or a target binding site of an agent that address the shortcomings encountered in prior art methods, ideally in a high-throughput manner.

Against that background, the present inventors have developed a new (and surprisingly effective) approach for identifying, characterising and/or designing an agent or a target binding site of an agent, which addresses the above problems.

As discussed in detail below, the inventors' approach provides a method for epitope mapping that combines high resolution output with high-throughput, and represents an improved approach that addresses the structure versus epitope problem encountered in known methods. The inventors have surprisingly found that by normalising the screening data obtained using a control data set (for example, either using a conserved peptide/sequence, or using an agent targeting another epitope in the antigen of interest) it is possible to more accurately identify the amino acids that actually form the epitope recognised by an agent of interest.

Put another way, by using such a control, the inventors' method makes it possible to identify and exclude from the analysis the amino acid positions which are critical for overall structure of the antigen. By excluding those from the analysis, the remaining amino acid positions in the antigen that are found to influence antibody/agent binding are therefore part of the epitope.

Thus, advantageously, the method of the present invention is able to differentiate between amino acids that form part of the epitope of an antigen recognised by an agent and amino acids that are critical for overall structure. Specifically, an amino acid critical for overall structure will likely produce a high signal in both the experimental and control data sets and so such signals can be cancelled out in the normalised data set. In addition, the method of the present invention also advantageously allows for the omission of the need to use a SA value cut-off to arbitrarily filter possible non-epitope amino acids from the experimental output and so reduces the possibility of important amino acids being inadvertently excluded from the identified epitope (i.e. as false negatives). Other advantages of the present invention include the ability to use a smaller amount of agent (e.g. antibody) during the screening process and that purified antigen is not required for screening. In addition, the method of the present invention has high-throughput capacity, and so it is suitable for analysing tens or even hundreds of agents in parallel.

In a first aspect, the invention provides a method for identifying and/or characterising the target binding site of an agent, or fragment thereof, the method comprising:
(i) providing an agent or fragment thereof to be tested;
(ii) providing a library of displayed polypeptides, wherein the library comprises a plurality of polypeptide molecules having different sequences derived from the target of the agent or fragment thereof;
(iii) screening the library for polypeptide molecules to which the agent or fragment thereof binds, and identifying a first population of polypeptide molecules to which the agent or fragment thereof binds and/or a second population of polypeptide molecules to which the agent or fragment thereof does not bind;
(iv) determining the sequences of the first and/or second population of polypeptide molecules;
(v) identifying and/or characterising the target binding site of the agent or fragment thereof from the information obtained in step (iv);
   wherein polypeptides that are not properly displayed are excluded from the step of identifying and/or characterising the target binding site in step (v).

In a second aspect, the invention provides a method for identifying a target binding site of an agent or fragment thereof and/or designing a target binding site to which binding of an agent or fragment thereof is increased or decreased, the method comprising:
(i) providing an agent or fragment thereof to be tested;
(ii) providing a library of displayed polypeptides, wherein the library comprises a plurality of polypeptide molecules having different sequences derived from the target of the agent or fragment thereof;
(iii) screening the library for polypeptide molecules to which the agent or fragment thereof binds, and identifying a first population of polypeptide molecules to which the agent or fragment thereof binds and/or a second population of polypeptide molecules to which the agent or fragment thereof does not bind;
(iv) determining the sequences of the first and/or second population of polypeptide molecules;
(v) identifying a target binding site of the agent or fragment thereof and/or designing a target binding site to which binding of the agent or fragment thereof is increased or decreased, from the information obtained in step (iv);
   wherein polypeptides that are not properly displayed are excluded from the step of identifying and/or designing an epitope and/or a target binding site in step (v).

By “target binding site” we include the meaning of a region on a protein that is bound by another molecule or ligand with specificity (i.e. not non-specific binding). For example, the target binding site on a protein molecule may be the region of that protein that is specifically recognised by an antibody, another protein, small molecule, or nucleic acid aptamer.

In some embodiments of the first and second aspects, the polypeptides are not properly displayed due to: (i) the polypeptides being mis-folded, and/or (ii) due to the polypeptides not being expressed or being expressed at a level insufficient for proper display, and/or (iii) due to the polypeptides being degraded, and/or (iv) or due to a combination thereof.

By “mis-folded” we include the meaning of a protein that is not able to achieve its native conformation when in solution, which may be characterised by the protein not exhibiting its expected structural or functional properties. For example, a mis-folded protein may lack expected enzymatic activity, may become less soluble or insoluble, may form aggregate structures, and/or may be degraded by the cellular machinery of a host organism (e.g. proteolysis in a lysosome or by the proteasome). In addition, a protein may become misfolded (partly or in its entirety) as a result of attaching to a surface, like a plastic surface.

By “not expressed or are expressed at a level insufficient for proper display” we include the meaning of a polypeptide that is expressed at a level that does not allow for its display in a biopanning experiment. The lack of expression may be caused by disruption of transcription from a corresponding polynucleotide sequence, a block in translation of the polypeptide by a ribosome, or it may be due to mis-folding of the polypeptide after translation leading to issues to insolubility, aggregation, and/or degradation of the polypeptide.

By “degraded” we include the meaning of a polypeptide that has been broken down into smaller polypeptides or amino acids by any mechanism of proteolysis. The proteolysis mechanism may be catalytic (e.g. caused by protease enzymes or by autoproteolysis) or non-catalytic processes (e.g. caused by elevated temperature, acid hydrolysis, or alkaline hydrolysis). In situations where a polypeptide is expressed in a host cell, the degradation of a polypeptide carried out in a lysosome or by the ubiquitin-dependent proteasome pathway.

In some embodiments of the first and second aspects, the plurality of polypeptide molecules is encoded by a plurality of polynucleotides.

When the plurality of polypeptides is encoded by a plurality of polynucleotides, a link is created between phenotype and genotype in each polypeptide in the displayed library (i.e. the exact polypeptide sequence displayed can be determined by sequencing the associated polynucleotide).

In some embodiments of the first and second aspects, the plurality of polypeptides comprises polypeptides that are 5 to 1000 amino acids in length, 25 to 950 amino acids in length, 50 to 900 amino acids in length, 100 to 850 amino acids in length, 150 to 800 amino acids in length, 200 to 750 amino acids in length, 250 to 700 amino acids in length, 300 to 650 amino acids in length, 350 to 600 amino acids in length, 400 to 550 amino acids in length, or 450 to 500 amino acids in length. Preferably, the plurality of polypeptides comprises polypeptides that are 50 to 700 amino acids in length, more preferably 50 to 500 amino acids in length, most preferably 50 to 300 amino acids in length.

In some embodiments of the first and second aspects, the different sequences are derived from a reference sequence of the target of the agent or fragment thereof.

By “reference sequence” we include the meaning of the non-mutated/wildtype amino acid sequence of the members of the displayed polypeptide library (or the non-mutated/wildtype nucleotide sequence of a plurality of polynucleotides encoding such a library) prior to the introduction of amino acid (or nucleotide) changes. All changes introduced to members of the displayed library are defined relative to the original, reference sequence for that library. For instance, the reference sequence may be the wildtype sequence of a protein (or polynucleotide encoding that protein) prior to the introduction of substitutions during library production, and all substitutions are expressed as changes relative to the wildtype sequence.

In some embodiments of the first and second aspects, the target binding site is a conformational binding site or is a linear binding site.

In some embodiments of the first and second aspects, one or more amino acid of the target binding site is identified and/or characterised. In some embodiments, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more amino acids of the agent or fragment thereof are identified and/or characterised.

In some embodiments of the first and second aspects, the identified target binding site is 1 to 20 amino acids, 2 to 19 amino acids, 3 to 18 amino acids, 4 to 17 amino acids, 5 to 16 amino acids, 6 to 15 amino acids, 7 to 14 amino acids, 8 to 13 amino acids, 9 to 12 amino acids, 10 to 11 amino acids, 11 to 20 amino acids, 12 to 20 amino acids, 13 to 20 amino acids, 14 to 20 amino acids, 15 to 20 amino acids, 16 to 20 amino acids, 17 to 20 amino acids, 18 to 20 amino acids, or 19 to 20 amino acids. Preferably the identified target binding site is 1 to 12 amino acids, more preferably 2 to 12 amino acids, most preferably 4 to 10 amino acids.

In some embodiments of the first and second aspects, the agent is one or more selected from the group comprising: an antibody or fragment thereof, a protein or fragment thereof, a carbohydrate or fragment thereof, a lipid or fragment thereof, a nucleic acid or fragment thereof, or a small molecule or fragment thereof, and combinations thereof.

By “small molecule” we include the meaning of an organic or inorganic molecule that typically has a molecular weight of ≤1000 Daltons.

In some embodiments of the first and second aspects, the protein agent is a receptor protein, a cytokine, a hormone, an enzyme, a nucleic acid binding protein, a storage protein, a structural protein, a lectin, or a transport protein.

In some embodiments of the first and second aspects, the nucleic acid agent is a nucleic acid aptamer (e.g. a DNA aptamer or an RNA aptamer), genomic DNA, single stranded DNA (ssDNA), double stranded DNA (dsDNA), plasmid or vector DNA, single stranded RNA (ssRNA), double stranded RNA (dsRNA), transfer RNA (tRNA), ribosomal RNA (rRNA), short interfering RNA (siRNA), micro RNA (miRNA), an antisense oligonucleotide, crispr RNA (crRNA), trans-acting crispr RNA (tracrRNA), guide RNA (gRNA), single guide RNA (sgRNA), non-coding RNA (ncRNA), long non-coding RNA (lncRNA), or small non-coding RNA (sncRNA).

In some embodiments of the first and second aspects, the carbohydrate agent is one or more selected from the group comprising: monosaccharides, disaccharides, oligosaccharides, and polysaccharides.

In some embodiments of the first and second aspects, the lipid agent is one or more selected from the group comprising: fatty acyls, glycerides, phospholipids, sphingolipids, sterols, prenols, glycolipids and polyketides.

In some embodiments of the first and second aspects, the agent is provided as part of a biological sample or is derived from a biological sample. In some embodiments, the biological sample is one or more selected from the group comprising: blood, serum, plasma, urine, saliva, cerebrospinal fluid (CSF), a virology swab, a biopsy sample, and combinations thereof. Preferably, the biological sample is serum.

Biological samples (particularly, blood and serum) from a subject may be used in the methods of the present invention to identify epitopes on self-proteins of that subject that are recognised by antibodies implicated in autoimmune responses and diseases/disorders caused by such responses. Identification of epitopes recognised by autoantibodies may subsequently be used to predict disease progression, determine the best treatment for an individual, develop disease prevention strategies, understand the cause/trigger for the autoimmune response, and allow design of therapies based on the epitope information obtained.

Furthermore, biological samples from a subject may be used in the method of the present invention to identify epitopes that are recognised by antibodies produced in that subject in response to a viral, bacterial or other pathogen infection or following administration of a vaccine. Identification of such epitopes can be used to correlate immune protection conferred by antibodies to a particular epitope with diseases outcome and aid the design of efficient vaccines that trigger the production of antibodies targeting the most clinically relevant epitopes. In addition, therapeutic human monoclonal antibodies may be used to target such clinically relevant epitopes, such as a single antibody, a bispecific antibody, or a mixture of two or more monoclonal antibodies.

In some embodiments of the first and second aspects, the biological sample comprises one or more autoantibody.

By “autoantibody” we include the meaning of an antibody that is directed against one or more of the individual's own proteins.

In some embodiments of the first and second aspects, the target binding site is an epitope. In some embodiments, the epitope is a conformational epitope, a linear epitope, or a discontinuous epitope.

By “epitope” we include the meaning of the part(s) of a target molecule that is/are recognised/bound by an antibody or other specifically targeting agent (e.g. small molecule or aptamer). In the case of a protein target, the epitope is the combination of amino acids that is recognised/bound by the antibody/agent, for example, a conformational epitope, a linear epitope, or a discontinuous epitope.

In a preferred embodiment of the first and second aspects, the agent is an antibody or fragment thereof, and the target binding site is the antibody epitope.

In some embodiments of the first and second aspects, the library of displayed polypeptides in step (ii) does not comprise the complete and/or full-length target of the agent or fragment thereof.

In some embodiments of the first and second aspects, polypeptides that are not properly displayed are excluded from the step of identifying and/or characterising the target binding site in step (v) by:
excluding polypeptides that are not properly displayed from the library; and/or
excluding polypeptides that are not properly displayed from the first and/or second population of polypeptide molecules; and/or
excluding information about polypeptides that are not properly displayed from the information in step (iv).

In some embodiments of the first and second aspects, the method comprises the further step of identifying polypeptides that are not properly displayed using a control agent that is capable of selectively binding to properly displayed polypeptides. In some embodiments, the control agent that is capable of selectively binding to properly displayed polypeptides comprises a control antibody that selectively recognises the target.

By “control agent” we include the meaning of an agent that is able to selectively bind to a properly displayed polypeptide/agent but is unable to bind to a polypeptide/agent that is not properly displayed.

By “control antibody” we include the meaning of an antibody that is able to selectively bind to a properly displayed polypeptide/agent but is unable to bind to a polypeptide/agent that is not properly displayed.

In some embodiments of the first and second aspects, each polypeptide in the library comprises a control peptide which is indicative of a properly displayed polypeptide.

By “control peptide” we include the meaning of a peptide sequence that is present in all members of the displayed library and is indicative of proper display of a peptide. A control peptide may be a naturally occurring peptide sequence that is shared by all members of displayed library, or it may be an exogenous peptide sequence that is introduced to all members of the displayed library and not subject to mutation during library generation (e.g. a peptide tag). A control peptide may be subjected to mutagenesis during library generation, or it may not be subjected to mutagenesis during library generation (i.e. its sequence is fully conserved in all members of the displayed library).

In some embodiments of the first and second aspects, the control peptide is a peptide that is shared by all polypeptides in the library of displayed polypeptides.

In some embodiments of the first and second aspects, the control peptide is an exogenous peptide sequence that is introduced to all polypeptides in the library of the displayed polypeptides. In some embodiments, the control peptide is a DYKDDDDK peptide tag, a c-Myc-tag, a HA-tag, a V5-tag, or a His-tag.

In some embodiments of the first and second aspects, the control peptide is not subject to mutagenesis during generation of the library of displayed polypeptides.

In some embodiments of the first and second aspects, the agent capable of selectively binding to properly displayed polypeptides comprises a control antibody that selectively recognises a control peptide.

In some embodiments of the first and second aspects, step (ii) comprises introducing polynucleotide sequences encoding the plurality of polypeptide molecules into a host system capable of displaying the polypeptides and expressing the polypeptides in the host system.

In some embodiments of the first and second aspects, step (iii) comprises:
(a) incubating the library of displayed polypeptides with the agent or fragment thereof;
(b) immobilising the agent or fragment thereof onto a solid support;
(c) washing the solid support to remove unbound displayed polypeptides to obtain the second population of polypeptide molecules; and,
(d) eluting the bound displayed polypeptides to obtain the first population of polypeptide molecules.

In some embodiments of the first and second aspects, step (iii) comprises:
(a) immobilising the agent or fragment thereof onto a solid support;
(b) incubating the library of displayed polypeptides with the agent or fragment thereof;
(c) washing the solid support to remove unbound displayed polypeptides to obtain the second population of polypeptide molecules; and,
(d) eluting the bound displayed polypeptides to obtain the first population of polypeptide molecules.

In some embodiments of the first and second aspects, step (iii) is repeated prior to the start of step (iv). In some embodiments, step (iii) is repeated once, twice, three times, or four times prior to the start of step (iv).

In some embodiments of the first and second aspects, step (iii) further comprises performing at least one control screening of the library of displayed polypeptides. Preferably, the control screening of the library of displayed polypeptides is performed in parallel with the step of screening the library for polypeptide molecules to which the agent or fragment thereof binds. It will be appreciated that could be done by, for example, dividing the library of displayed polypeptides into two identical portions (in which each portion contains the same library members), and performing the test screening on one portion and the control screening on the other.

By “control screening” we include the meaning of a screening that does not include the molecule to be tested in step (i) (i.e. agent or fragment thereof in the first and second aspects or antigen or fragment thereof in the third aspect).

In some embodiments, the at least one control screening comprises screening the library of displayed polypeptides against a second agent or fragment thereof.

It will be appreciated that the second agent (for example, an antibody) may be an agent that binds to a different antigen, an agent that binds to the same antigen at a different site, or an agent that recognises a control peptide present in members of the library of displayed polypeptides (for example, an anti-DYKDDDDK antibody).

In some embodiments of the first and second aspects, step (iii) further comprises screening the library of displayed polypeptides against a second agent, wherein the second agent is an agent or fragment thereof that recognises a different target molecule to the target molecule recognised by the agent to be tested.

In some embodiments of the first and second aspects, step (iii) further comprises screening the library of displayed polypeptides against a second agent, wherein the second agent or fragment thereof is an agent or fragment thereof that recognises a different target binding site in the same target molecule recognised by the agent to be tested. In some embodiments, the different target binding site is a different epitope in the same target molecule.

In some embodiments of the first and second aspects, step (iii) further comprises screening the library of displayed polypeptides against a second agent, wherein the second agent or fragment thereof is an agent or fragment thereof that recognises a control peptide that is present in the members of the library of displayed polypeptides.

In some embodiments of the first and second aspects, the agent is one or more selected from the group comprising: an antibody or fragment thereof, a protein or fragment thereof, a carbohydrate or fragment thereof, a lipid or fragment thereof, a nucleic acid or fragment thereof, or a small molecule or fragment thereof, and combinations thereof. In preferred embodiments, the second agent is an antibody or fragment thereof.

In some embodiments of the first and second aspects, the library of displayed polypeptides is displayed on the outer surface of one or more virus particle, on the outer surface of one or more cell, as part of a ribosome display system, as part of a DNA display system, or as part of an mRNA display system.

When cell or virus-based display systems are used, the polynucleotides encoding the library are introduced to the cells/viruses and the library is produced in and displayed on the cells/viruses into which the polynucleotides were introduced. In the case of ribosome display, DNA display, or mRNA display systems, the library members are produced by in vitro transcription/translation and displayed in conjunction with the corresponding mRNA-ribosome complex, DNA, or mRNA.

In some embodiments of the first and second aspects, the library of displayed polypeptides is displayed on the outer surface of one or more virus particle. In some embodiments, the one or more virus particle is one or more selected from the group comprising: bacteriophage, baculovirus, lentivirus, adenovirus, tobacco mosaic virus, or avian leukosis virus. Examples of the use of baculovirus (Makela and Oker-Blom, 2006. Adv. Virus Res., 68:91-112), lentivirus (Taube et al, 2008. PLOS One, 3(9):e3181), adenovirus (Waterkamp et al, 2006. J. Gene Med., 8(11):1307-1319), tobacco mosaic virus (Smith et al, 2009. Curr. Top. Microbiol. Immunol., 332:13-31), and avian leukosis virus (Yu et al, 2015. Proc. Natl. Acad. Sci. USA, 112(32):9860-9865) are known to the skilled person.

In preferred embodiments, the one or more virus particle is a bacteriophage is one or more selected from the group comprising: M13 bacteriophage, Fd bacteriophage, T4 bacteriophage, or T7 bacteriophage. More preferably, the bacteriophage is M13 bacteriophage.

In some embodiments of the first and second aspects, the library of displayed polypeptides is displayed on the outer surface of one or more cell. In some embodiments, the one or more cell is one or more selected from the group comprising: a yeast cell, a mammalian cell, an insect cell, and a bacterial cell.

In some embodiments of the first and second aspects, the plurality of polypeptide molecules is obtained by mutagenesis of the target.

In some embodiments of the first and second aspects, the library comprises 1,000 or more, 2,000 or more, 3,000 or more, 4,000 or more, 5,000 or more, 6,000 or more, 7,000 or more, 8,000 or more, 9,000 or more, 10,000 or more, 25,000 or more, 50,000 or more, 100,000 or more, 250,000 or more, 500,000 or more, 750,000 or more, 1,000,000 or more, 5,000,000 or more, 10,000,000 or more, 100,000,000 or more, 1,000,000,000 or more, 10,000,000,000 or more, 100,000,000,000 or more different polypeptide sequences, or 100,000,000,000,000 or more different polypeptide sequences.

In some embodiments of the first and second aspects, each of the plurality of polypeptide molecules comprises between 1 to 100 amino acid changes, preferably between 1 to 50 amino acid changes, more preferably between 1 to 10 amino acid changes, or most preferably between 1 to 3 amino acid changes, compared to the target of the agent or fragment thereof.

In some embodiments of the first and second aspects, the amino acid change is to any of the twenty natural amino acids, or unnatural amino acids using cells with engineered tRNA.

In many known epitope mapping approaches the amino acid changes introduce an alanine at each substituted position in the antigen. By instead introducing substitutions of any natural amino acid, it is possible determine what amino acids or types of amino acids are tolerated/not tolerated at a given position. In turn, the chemical properties of tolerated/not tolerated substitutions can provide details of how the actual interaction between the antigen and the antibody/agent occurs.

In some embodiments of the first and second aspects, the amino acid changes introduced to the target cover all of the positions of the amino acid sequence of the target.

In some embodiments of the first and second aspects, the amino acid changes introduced to the target cover a portion of the positions of the amino acid sequence of the target. In some embodiments of the first and second aspects, the portion of the amino acid sequence of the target covered is a domain or sub-domain of the target.

In some embodiments of the first and second aspects, mutagenesis is performed by error-prone PCR, propagation of a polynucleotide sequence encoding the target in a bacterial mutator strain, or site-directed mutagenesis.

In some embodiments of the first and second aspects, the first population of polypeptide molecules is screened for polypeptide molecules to which the agent or fragment thereof binds, to thereby identify a third population of polypeptide molecules and/or a fourth population of polypeptide molecules to which the agent or fragment thereof does not bind.

In some embodiments of the first and second aspects, the second population of polypeptide molecules is screened for polypeptide molecules to which the agent or fragment thereof binds to thereby identify a third population of polypeptide molecules and/or a fourth population of polypeptide molecules to which the agent or fragment thereof does not bind.

In some embodiments of the first and second aspects, step (iv) comprises:
(a) amplifying the plurality of polynucleotides encoding the first and/or second population of polypeptide molecules to produce a first and/or second population of amplified polynucleotides; and,
(b) performing sequencing on the first and/or second population of amplified polynucleotides.

In some embodiments of the first and second aspects, step (v) comprises comparing the information obtained in step (iv) for the first population of polypeptide molecules with information obtained for at least one reference population of polypeptides.

By “reference population of polypeptides” we include the meaning of a population of polypeptides derived from the same library of displayed polypeptides used in step (ii) against which information obtained from step (iv) for the first and/or second population of polypeptides may be compared. The information relating to the reference population of polypeptides can be used by a user to normalise the information obtained for the first and/or second population of polypeptides. For example, information that is found to be the same in both the reference population of polypeptides and the first/second population of polypeptides can be removed from the analysis carried out in step (v).

In some embodiments of the first and second aspects, the at least one reference population of polypeptides is obtained from a control screening of the library of displayed polypeptides.

In some embodiments of the first and second aspects, the at least one reference population of polypeptides is:
(a) the library of displayed polypeptides prior to screening; and/or,
(b) the second population of polypeptide molecules identified in step (iii); and/or,
(c) a population of polypeptides obtained by screening the library of displayed polypeptides against a second agent or fragment thereof.

In some embodiments of the first and second aspects, the second agent is an agent or fragment thereof that recognises a different target molecule to the target molecule recognised by the agent to be tested.

In some embodiments of the first and second aspects, the second agent is an agent or fragment thereof that recognises a different target binding site in the same target molecule recognised by the agent to be tested. In some embodiments, the different target binding site is a different epitope in the same target molecule.

In some embodiments of the first and second aspects, the second agent is an agent or fragment thereof that recognises a control peptide that is present in the members of the library of displayed polypeptides.

In some embodiments of the first and second aspects, step (v) comprises:
(a) aligning the sequences of the first population of polypeptide molecules and/or reference population of polypeptides with the reference sequence of the target of the agent or fragment thereof;
(b) counting the frequency of adenine, thymine, guanine, and cytosine residues present at each position in the polynucleotides encoding the first population polypeptides and/or reference population of polypeptides relative to the reference sequence of the target of the agent or fragment thereof; and,
(c) converting the counts obtained in sub-step (b) into counts of amino acid residues for each position in the encoded polypeptides in order to obtain frequencies of amino acids at each position in the first population polypeptides and/or reference population of polypeptides.

In some embodiments of the first and second aspects, step (v) further comprises:
predicting the solvent accessibility for each amino acid in the target of the agent or fragment thereof for the first population polypeptides and/or reference population of polypeptides.

By “solvent accessibility” we include the meaning of the surface area of a biomolecule that is accessible to a solvent. This is also referred to in the field as accessible surface area (ASA) or solvent-accessible surface area (SASA). Solvent accessibility is typically based on measurements of a square angstrom (Å²) area of a molecule or moiety within a molecule that that is in contact with a solvent. In the case of a protein, the solvent accessibility of each amino acid in that protein can be measured and can be used an indicator of whether the amino acid is likely to be on the surface of the protein (i.e. more accessible to solvent) or buried within the core of the protein (i.e. less accessible to solvent).

In some embodiments of the first and second aspects, step (v) further comprises:
determining the variability at each amino acid position in the target or fragment thereof for the first population polypeptides and/or reference population of polypeptides based on the amino acid frequencies obtained in sub-step (c).

In some embodiments of the first and second aspect, step (v) comprises determining the variability at each amino acid position using Shannon entropy, Rao's quadratic entropy, or mutation frequency.

In some embodiments of the first and second aspects, step (v) further comprises:
identifying and/or characterising the target binding site of the agent or fragment thereof based on the ratio of the variability at each amino acid position determined for the first population of polypeptides and the variability at each amino acid position determined for the reference population of polypeptides.

In relation to the ratio of the variability at a particular amino acid position for the first population polypeptides and the reference population of polypeptides, a ratio value of 1.0 indicates that for that position there is no difference in variability between the two population of polypeptides after screening and suggests that the position is not likely to be part of the epitope on the target that is recognised by the tested agent. A ratio value of >1.0 indicates that for that amino acid position the variability is lower in the first population of polypeptides and so suggests that that position could be part of the epitope. In a typical comparison, the top five amino acid positions with the highest ratios may have a ratio between 2.0 and 8.0 and positions six to ten between 2.0 to 1.1. However, the affinity between an agent and a target molecule can influence the ranges of ratio observed. For instance, where the affinity between an agent and a target molecule is strong, higher top ratios may be observed (e.g. >8.0). In contrast, where the affinity between an agent and a target molecule is weak, lower top ratios may be observed (e.g. 2.5).

In some embodiments of the first and second aspects, an amino acid forming part of the target binding site has a ratio of the variability at an amino acid position for the first population of polypeptides and the reference population of polypeptides of >1.0, ≥1.1, ≥1.2, ≥1.3, ≥1.4, ≥1.5, ≥1.6, ≥1.7, ≥1.8, ≥1.9, ≥2.0, ≥2.5, ≥3.0, ≥3.5, ≥4.0, ≥4.5, ≥5.0, ≥5.5, ≥6.0, ≥7.0, ≥7.5, ≥8.0, ≥8.5, ≥9.0, ≥9.5, ≥10.0, ≥11.0, ≥12.0, ≥13.0, ≥14.0, ≥15.0, ≥16.0, ≥17.0, ≥18.0, ≥19.0, ≥20.0, ≥21.0, ≥22.0, ≥23.0, ≥24.0, ≥25.0, ≥26.0, ≥27.0, ≥28.0, ≥29.0, or ≥30.0. Preferably, an amino acid forming part of the target binding site has a ratio of the variability for the first population of polypeptides and the reference population of polypeptides is ≥1.1, more preferably ≥2.0, yet more preferably ≥5.0, most preferably ≥8.0.

In some embodiments of the first and second aspects, an amino acid forming part of the target binding site has a ratio of the variability at an amino acid position for the first population polypeptides and the reference population of polypeptides of between 1.0 and 30.0, between 1.1 and 29.0, between 1.3 and 28.0, between 1.4 and 27.0, between 1.5 and 26.0, between 1.6 and 25.0, between 1.7 and 24.0, between 1.8 and 23.0, between 1.9 and 22.0, between 2.0 and 21.0, between 2.5 and 20.0, between 3.0 and 19.0, between 3.5 and 18.0, between 4.0 and 17.0, between 4.5 and 16.0, between 5.0 and 14.0, between 6.0 and 13.0, between 6.5 and 12.0, between 7.0 and 11.0, between 7.5 and 10.0, between 8.0 and 9.0, between 8.5 and 30.0, between 9.0 and 30.0, between 9.5 and 30.0, between 10.0 and 30.0, between 11.0 and 30.0, between 12.0 and 30.0, between 13.0 and 30.0, between 14.0 and 30.0, between 15.0 and 30.0, between 16.0 and 30.0, between 17.0 and 30.0, between 18.0 and 30.0, between 19.0 and 30.0, between 20.0 and 30.0, between 21.0 and 30.0, between 22.0 and 30.0, between 23.0 and 30.0, between 24.0 and 30.0, between 25.0 and 30.0, between 26.0 and 30.0, between 27.0 and 30.0, between 28.0 and 30.0, or between 29.0 and 30.0. Preferably, an amino acid forming part of the target binding site has a ratio of the variability an amino acid position for the first population polypeptides and the reference population of polypeptides is between 1.1 and 30.0, more preferably between 1.1 and 15.0, more preferably between 2.0 and 8.0, and yet more preferably between 2.0 and 15.0.

A ratio value of <1.0 indicates that the variation at an amino acid position is increased in the first population of polypeptides. In those cases, the frequency of the wildtype amino acid is lower, and another non-wildtype amino acid is enriched (thereby leading to an overall increase in variability at that position). Such enrichment of a non-wildtype amino acid at a particular position could suggest that the variant polypeptide having that amino acid change binds more strongly to the tested agent.

By “non-wildtype amino acid” we include the meaning of any amino acid other than the amino acid present at that position in the corresponding reference polypeptide/agent.

In some embodiments of the first and second aspects, the ratio of the variability at an amino acid position for the first population of polypeptides and the reference population of polypeptides is <1.0. In some embodiments, the ratio of the variability at an amino acid position for the first population of agents and the reference population of agents is ≤0.95, ≤0.90, ≤0.85, ≤0.80, ≤0.75, ≤0.70, ≤0.65, ≤0.60, ≤0.55, ≤0.50, ≤0.45, ≤0.40, ≤0.35, ≤0.30, ≤0.25, ≤0.20, ≤0.15, ≤0.10, ≤0.05, ≤0.025, or ≤0.01.

In some embodiments of the first and second aspects, when an amino acid position has a ratio of the variability at an amino acid position for the first population of polypeptides and the reference population of polypeptides of <1.0, the first population of polypeptides is enriched for agents having a non-wildtype amino acid at that position.

In some embodiments of the first and second aspects, the one or more non-wildtype amino acid is enriched at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, at least 10.0-fold, at least 11.0-fold, at least 12.0-fold, at least 13.0-fold, at least 14.0-fold, at least 15.0-fold, at least 16.0-fold, at least 17.0-fold, at least 18.0-fold, at least 19.0-fold, or at least 20.0-fold relative to the wildtype amino acid at that position.

In some embodiments of the second aspect, the binding of a polypeptide to the agent is increased relative to the binding of a polypeptide having the reference polypeptide sequence to the agent.

In some embodiments of the second aspect, the binding of a polypeptide to the agent is decreased relative to the binding of a polypeptide having the reference polypeptide sequence to the agent.

Such an increase/decrease of binding affinity could be quantified using surface-plasmon-resonance (SPR), inhibition ELISA, or equilibrium dialysis where the equilibrium dissociation constant KD is measured.

In a third aspect, the invention provides a method for identifying an agent or fragment thereof and/or designing an agent or fragment thereof, to which binding of a target antigen or fragment thereof, is increased or decreased, the method comprising:
(i) providing a target antigen or fragment thereof to be tested;
(ii) providing a library of displayed agents, wherein the library comprises a plurality of agent molecules or fragments thereof having different sequences derived from the agent or fragment thereof;
(iii) screening the library for agent molecules to which the antigen or fragment thereof binds, and identifying a first population of agent molecules to which the antigen or fragment thereof binds and/or a second population of agent molecules to which the antigen or fragment thereof does not bind;
(iv) determining the sequences of the first and/or second population of agent molecules;
(v) identifying and/or designing an agent or fragment thereof to which binding of the antigen or fragment thereof is increased or decreased, from the information obtained in step (iv);
   wherein agent molecules that are not properly displayed are excluded from the step of identifying and/or designing an agent or fragment thereof in step (v).

In some embodiments of the third aspect, the agents are not properly displayed due to the agents being mis-folded, due to the agents not being expressed or being expressed at a level insufficient for proper display, due to the agents being degraded, or due to a combination thereof.

In some embodiments of the third aspect, the plurality of agent molecules comprises a plurality of polypeptides. In some embodiments of the third aspect, the plurality of agents is encoded by a plurality of polynucleotides.

In some embodiments of the third aspect, the different sequences are derived from a reference sequence of the agent or fragment thereof.

In some embodiments of the third aspect, the binding of an agent to the antigen is increased relative to the binding of an agent having the reference agent sequence to the antigen.

In some embodiments of the third aspect, the binding of an agent to the antigen is decreased relative to the binding of an agent having the reference agent sequence to the antigen.

Such an increase/decrease of binding affinity could be quantified using surface-plasmon-resonance (SPR), inhibition ELISA, or equilibrium dialysis where the equilibrium dissociation constant KD is measured.

In some embodiments of the third aspect, the agent or fragment thereof is an antibody or fragment thereof. In some embodiments, the target antigen comprises the antibody epitope.

In some embodiments of the third aspect, one or more amino acid of the agent or fragment thereof is identified and/or characterised.

In some embodiments of the third aspect, 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more amino acids of the agent or fragment thereof are identified and/or characterised.

In some embodiments of the third aspect, the antigen or fragment thereof is one or more selected from the group comprising: a protein or fragment thereof, an antibody or fragment thereof, a carbohydrate or fragment thereof, a lipid or fragment thereof, a nucleic acid or fragment thereof, or a small molecule or fragment thereof, or combinations thereof.

In some embodiments of the third aspect, the protein antigen is a receptor protein, a cytokine, a hormone, an enzyme, a nucleic acid binding protein, a storage protein, a structural protein, a lectin, an antibody or fragment thereof, or a transport protein.

In some embodiments of the third aspect, the nucleic acid antigen is a nucleic acid aptamer (e.g. a DNA aptamer or an RNA aptamer), genomic DNA, single stranded DNA (ssDNA), double stranded DNA (dsDNA), plasmid or vector DNA, single stranded RNA (ssRNA), double stranded RNA (dsRNA), transfer RNA (tRNA), ribosomal RNA (rRNA), short interfering RNA (SIRNA), micro RNA (miRNA), an antisense oligonucleotide, crispr RNA (crRNA), trans-acting crispr RNA (tracrRNA), guide RNA (gRNA), single guide RNA (sgRNA), non-coding RNA (ncRNA), long non-coding RNA (lncRNA), or small non-coding RNA (sncRNA).

In some embodiments of the third aspect, the carbohydrate antigen is one or more selected from the group comprising: monosaccharides, disaccharides, oligosaccharides, and polysaccharides.

In some embodiments of the third aspect, the lipid antigen is one or more selected from the group comprising: fatty acyls, glycerides, phospholipids, sphingolipids, sterols, prenols, glycolipids and polyketides.

In some embodiments of the third aspect, the antigen or fragment thereof is provided as part of a biological sample or is derived from a biological sample. In some embodiments, the biological sample is one or more selected from the group comprising: blood, serum, plasma, urine, saliva, cerebrospinal fluid (CSF), a virology swab, a biopsy sample, and combinations thereof.

In some embodiments of the third aspects, the biological sample comprises one or more autoantibody.

In a preferred embodiment of the third aspect, the agent is an antibody or fragment thereof, and the target binding site is the antibody epitope.

In some embodiments of the third aspect, the library of displayed agents in step (ii) does not comprise the complete and/or full-length agent or fragment thereof.

In some embodiments of the third aspect, agent molecules that are not properly displayed are excluded from the step of identifying and/or characterising the target binding site in step (v) by:
excluding agent molecules that are not properly displayed from the library; and/or
excluding agent molecules that are not properly displayed from the first and/or second population of polypeptide molecules; and/or
excluding information about agent molecules that are not properly displayed from the information in step (iv).

In some embodiments of the third aspect, the method comprises the further step of identifying agent molecules that are not properly displayed using an agent capable of selectively binding to properly displayed agent molecules. In some embodiments, the agent capable of selectively binding to properly displayed agent molecules comprises a control antibody that selectively recognises the agent.

In some embodiments of the third aspect, each agent in the library comprises a control peptide which is indicative of a properly displayed polypeptide.

In some embodiments of the third aspect, the control peptide is a peptide that is shared by all agents in the library of displayed agents.

In some embodiments of the third aspect, the control peptide is an exogenous peptide sequence that is introduced to all agents in the library of the displayed agents. In some embodiments, the control peptide is a DYKDDDDK peptide tag, a c-Myc-tag, a HA-tag, a V5-tag, or a His-tag.

In some embodiments of the third aspect, the control peptide is not subject to mutagenesis during generation of the library of displayed agents.

In some embodiments of the third aspect, the agent capable of selectively binding to properly displayed agents comprises a control antibody that selectively recognises a control peptide.

In some embodiments of the third aspect, step (ii) comprises introducing polynucleotide sequences encoding the plurality of agent molecules into a host system capable of displaying the polypeptides and expressing the agent molecules in the host system.

In some embodiments of the third aspect, step (iii) comprises:
(a) incubating the library of displayed agents with the target antigen or fragment thereof;
(b) immobilising the target antigen or fragment thereof onto a solid support;
(c) washing the solid support to remove unbound displayed agents to obtain the second population of agent molecules; and,
(d) eluting the bound displayed agents to obtain the first population of agent molecules.

In some embodiments of the third aspect, step (iii) comprises:
(a) immobilising the target antigen or fragment thereof onto a solid support;
(b) incubating the library of displayed agents with the target antigen or fragment thereof;
(c) washing the solid support to remove unbound displayed agents to obtain the second population of agent molecules; and,
(d) eluting the bound displayed agents to obtain the first population of agent molecules.

In some embodiments of the third aspect, step (iii) is repeated prior to the start of step (iv). In some embodiments, step (iii) is repeated once, twice, three times, or four times prior to the start of step (iv).

In some embodiments of the third aspect, step (iii) further comprises performing at least one control screening of the library of displayed agents. In some embodiments, the at least one control screening comprises screening the library of displayed agents against a second antigen or fragment thereof.

It will be appreciated that the second antigen may be an antigen that binds to a different agent, an antigen that binds to the same agent at a different site or via different mechanism, or an antigen that recognises a control peptide present in members of the library of displayed agents.

In some embodiments of the third aspect, step (iii) further comprises screening the library of displayed agents against a second antigen, wherein the second antigen is an antigen or fragment thereof that recognises an agent that is different to the agent recognised by the target antigen to be tested.

In some embodiments of the third aspect, step (iii) further comprises screening the library of displayed polypeptides against a second antigen, wherein the second antigen is an antigen or fragment thereof that recognises a different target binding site in the same agent recognised by the target antigen to be tested.

In some embodiments of the third aspect, step (iii) further comprises screening the library of displayed polypeptides against a second antigen, wherein the second antigen is an antigen or fragment thereof that recognises a control peptide that is present in the members of the library of displayed agents.

In some embodiments of the third aspect, the library of displayed agents is displayed on the outer surface of one or more virus particle, on the outer surface of one or more cell, as part of a ribosome display system, as part of a DNA display system, or as part of an mRNA display system.

In some embodiments of the third aspect, the library of displayed agents is displayed on the outer surface of one or more virus particle. In some embodiments, the one or more virus particle is one or more selected from the group comprising: bacteriophage, baculovirus, lentivirus, adenovirus, tobacco mosaic virus, and avian leukosis virus.

In preferred embodiments, the one or more virus particle is one or more bacteriophage selected from the group comprising: M13 bacteriophage, Fd bacteriophage, T4 bacteriophage, and T7 bacteriophage. More preferably, the bacteriophage is M13 bacteriophage.

In some embodiments of the third aspect, the library of displayed agents is displayed on the outer surface of one or more cell. In some embodiments, the one or more cell is one or more selected from the group comprising: a yeast cell, a mammalian cell, an insect cell, and a bacterial cell.

In some embodiments of the third aspect, the plurality of agent molecules is obtained by mutagenesis of the target.

In some embodiments of the third aspect, the library comprises 1,000 or more, 2,000 or more, 3,000 or more, 4,000 or more, 5,000 or more, 6,000 or more, 7,000 or more, 8,000 or more, 9,000 or more, 10,000 or more, 25,000 or more, 50,000 or more, 100,000 or more, 250,000 or more, 500,000 or more, 750,000 or more, 1,000,000 or more, 5,000,000 or more, 10,000,000 or more, 100,000,000 or more, 1,000,000,000 or more, 10,000,000,000 or more, 100,000,000,000 or more different polypeptide sequences, or 100,000,000,000,000 or more different polypeptide sequences.

In some embodiments of the third aspect, each of the plurality of agent molecules comprises between 1 to 100 amino acid changes, preferably between 1 to 50 amino acid changes, more preferably between 1 to 10 amino acid changes, or most preferably between 1 to 3 amino acid changes amino acid changes, compared to the target of the agent or fragment thereof.

In some embodiments of the third aspect, the amino acid change is to any of the twenty natural amino acids, or unnatural amino acids using cells with engineered tRNA.

In some embodiments of the third aspect, the amino acid changes introduced to the target cover all of the positions of the amino acid sequence of the target.

In some embodiments of the third aspect, the amino acid changes introduced to the target cover a portion of the positions of the amino acid sequence of the target. In some embodiments of the third aspect, the portion of the amino acid sequence of the target covered is a domain or sub-domain of the target.

In some embodiments of the third aspect, mutagenesis is performed by error-prone PCR, propagation of a polynucleotide sequence encoding the target in a bacterial mutator strain, or site-directed mutagenesis.

In some embodiments of the third aspect, the first population of agent molecules is screened for agent molecules to which the antigen or fragment thereof binds, to thereby identify a third population of agent molecules and/or a fourth population of agent molecules to which the agent or fragment thereof does not bind.

In some embodiments of the third aspect, the second population of agent molecules is screened for agent molecules to which the antigen or fragment thereof binds, to thereby identify a third population of agent molecules and/or a fourth population of agent molecules to which the agent or fragment thereof does not bind.

In some embodiments of the third aspect, step (iv) comprises:
(a) amplifying the plurality of polynucleotides encoding the first and/or second population of agent molecules to produce a first and/or second population of amplified polynucleotides; and,
(b) performing sequencing on the first and/or second population of amplified polynucleotides.

In some embodiments of the third aspect, step (v) comprises comparing the information obtained in step (iv) for the first population of agent molecules with information obtained for at least one reference population of agent molecules.

In some embodiments of the third aspect, the at least one reference population of agent molecules is obtained from a control screening of the library of displayed agents.

In some embodiments of the third aspect, the at least one reference population of agents is:
(a) the library of displayed agents prior to screening; and/or,
(b) the second population of agent molecules identified in step (iii); and/or,
(c) a population of agent molecules obtained by screening the library of displayed agents against a second antigen or fragment thereof.

In some embodiments of the third aspect, the second antigen is an antigen or fragment thereof that recognises an agent that is different to the agent recognised by the target antigen to be tested.

In some embodiments of the third aspect, the second antigen is an antigen or fragment thereof that recognises a different target binding site in the same agent recognised by the target antigen to be tested.

In some embodiments of the third aspect, the second antigen is an antigen or fragment thereof that recognises a control peptide that is present in the members of the library of displayed agents.

In some embodiments of the third aspect, step (v) comprises:
(a) aligning the sequences of the first population of agent molecules and/or reference population of agents with the reference sequence of the agent or fragment thereof;
(b) counting the frequency of adenine, thymine, guanine, and cytosine residues present at each position in the polynucleotides encoding the first population agents and/or reference population of agents relative to the reference sequence of the agent or fragment thereof; and,
(c) converting the counts obtained in sub-step (b) into counts of amino acid residues for each position in the displayed agent in order to obtain frequencies of amino acids at each position in the first population agents and/or reference population of agents.

In some embodiments of the third aspect, step (v) further comprises:
predicting the solvent accessibility for each amino acid in the target of the agent or fragment thereof for the first population polypeptides and/or reference population of polypeptides.

In some embodiments of the third aspect, step (v) further comprises:
determining the variability at each amino acid position in the target or fragment thereof for the first population agents and/or reference population of agents based on the amino acid frequencies obtained in sub-step (c).

In some embodiments of the third aspect, step (v) comprises determining the variability at each amino acid position using Shannon entropy, Rao's quadratic entropy, or mutation frequency.

In some embodiments of the third aspect, step (v) further comprises:
identifying and/or characterising the target binding site of the agent or fragment thereof based on the ratio of the variability at each amino acid position determined for the first population of agents and the variability at each amino acid position determined for the reference population of agents.

In relation to the ratio of the variability at a particular amino acid position for the first population agents and the reference population of agents, a ratio value of 1.0 indicates that for that position there is no difference in variability between the two population of agents after screening and suggests that the position is not likely to be part of the agent that recognises the tested antigen. A ratio value of >1.0 indicates that for that amino acid position the variability is lower in the first population of agents and so suggests that that position could be part of the epitope. In a typical comparison, the five amino acid positions with the highest ratios may have a ratio between 2.0 and 8.0 and positions six to ten between 2.0 to 1.1. However, the affinity between an agent and an antigen can influence the ranges of ratio observed. For instance, where the affinity between an agent and an antigen is strong, higher top ratios may be observed (e.g. >8.0). In contrast, where the affinity between an agent and an antigen is weak, lower top ratios may be observed (e.g. 2.5).

In some embodiments of the third aspect, an amino acid in the agent that recognises the antigen has a ratio of the variability at an amino acid position for the first population of agents and the reference population of agents of >1.0, ≥1.1, ≥1.2, ≥1.3, ≥1.4, ≥1.5, ≥1.6, ≥1.7, ≥1.8, ≥1.9, ≥2.0, ≥2.5, ≥3.0, ≥3.5, ≥4.0, ≥4.5, ≥5.0, ≥5.5, ≥6.0, ≥7.0, ≥7.5, ≥8.0, ≥8.5, ≥9.0, ≥9.5, ≥10.0, ≥11.0, ≥12.0, ≥13.0, ≥14.0, ≥15.0, ≥16.0, ≥17.0, ≥18.0, ≥19.0, ≥20.0, ≥21.0, ≥22.0, ≥23.0, ≥24.0, ≥25.0, ≥26.0, ≥27.0, ≥28.0, ≥29.0, or ≥30.0. Preferably, an amino acid in the agent that recognises the antigen has a ratio of the variability for the first population agents and the reference population of agents of ≥1.1, more preferably ≥2.0, yet more preferably ≥5.0, most preferably ≥8.0.Q

In some embodiments of the third aspect, an amino acid in the agent that recognises the antigen has a ratio of the variability at an amino acid position for the first population of agents and the reference population of agents of between 1.0 and 30.0, between 1.1 and 29.0, between 1.3 and 28.0, between 1.4 and 27.0, between 1.5 and 26.0, between 1.6 and 25.0, between 1.7 and 24.0, between 1.8 and 23.0, between 1.9 and 22.0, between 2.0 and 21.0, between 2.5 and 20.0, between 3.0 and 19.0, between 3.5 and 18.0, between 4.0 and 17.0, between 4.5 and 16.0, between 5.0 and 14.0, between 6.0 and 13.0, between 6.5 and 12.0, between 7.0 and 11.0, between 7.5 and 10.0, between 8.0 and 9.0, between 8.5 and 30.0, between 9.0 and 30.0, between 9.5 and 30.0, between 10.0 and 30.0, between 11.0 and 30.0, between 12.0 and 30.0, between 13.0 and 30.0, between 14.0 and 30.0, between 15.0 and 30.0, between 16.0 and 30.0, between 17.0 and 30.0, between 18.0 and 30.0, between 19.0 and 30.0, between 20.0 and 30.0, between 21.0 and 30.0, between 22.0 and 30.0, between 23.0 and 30.0, between 24.0 and 30.0, between 25.0 and 30.0, between 26.0 and 30.0, between 27.0 and 30.0, between 28.0 and 30.0, or between 29.0 and 30.0. Preferably, an amino acid in the agent that recognises the antigen has a ratio of the variability an amino acid position for the first population of agents and the reference population of agents of between 1.1 and 30.0, more preferably between 1.1 and 15.0, more preferably between 2.0 and 8.0, and yet more preferably between 2.0 and 15.0.

A ratio value of <1.0 indicates that the variation at an amino acid position is increased in the first population of agents. In those cases, the frequency of the wildtype amino acid is lower, and another non-wildtype amino acid is enriched (thereby leading to an overall increase in variability at that position). Enrichment of a non-wildtype amino acid at a particular position would suggest that the variant agent having that amino acid change binds more strongly to the tested antigen. Identifying enriched non-wildtype amino acids at particular positions in an agent would be particularly useful in antibody affinity maturation processes and any other processes that seek to identify agents with stronger or weaker affinity for an antigen including at different conditions such as specific pH, varied temperature, different salt concentrations or detergents.

In some embodiments of the third aspect, the ratio of the variability at an amino acid position for the first population of agents and the reference population of agents is <1.0. In some embodiments, the ratio of the variability at an amino acid position for the first population of agents and the reference population of agents is ≤0.95, ≤0.90, ≤0.85, ≤0.80, ≤0.75, ≤0.70, ≤0.65, ≤0.60, ≤0.55, ≤0.50, ≤0.45, ≤0.40, ≤0.35, ≤0.30, ≤0.25, ≤0.20, ≤0.15, ≤0.10, ≤0.05, ≤0.025, or ≤0.01.

In some embodiments of the third aspect, when an amino acid position has a ratio of the variability at an amino acid position for the first population of agents and the reference population of agents of <1.0, the first population of agents is enriched for agents having a non-wildtype amino acid at that position.

In some embodiments of the third aspect, the one or more non-wildtype amino acid is enriched at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, at least 10.0-fold, at least 11.0-fold, at least 12.0-fold, at least 13.0-fold, at least 14.0-fold, at least 15.0-fold, at least 16.0-fold, at least 17.0-fold, at least 18.0-fold, at least 19.0-fold, or at least 20.0-fold relative to the wildtype amino acid at that position.

In a fourth aspect, the invention provides a library of displayed polypeptides obtained using the method according to the first or second aspect.

In a fifth aspect, the invention provides a library of displayed agents obtained using the method according to the third aspect.

In a sixth aspect, the invention provides a method substantially as described herein with reference to the accompanying claims, description, examples and/or figures.

In a seventh aspect, the invention provides a library substantially as described herein with reference to the accompanying claims, description, examples and/or figures.

### DESCRIPTION OF THE FIGURES

Embodiments of the present invention will now be described, by way of example only, with reference to the accompanying figures, in which:

FIG. 1 shows a schematic diagram indicating an embodiment of the claimed method. Specifically, a phage display approach is used to identify antigen sequences that bind to a particular antibody molecule.

FIG. 2 shows the average ranking results for T470, E484, Y449, and F490 depending on the protocol used (E, F, G, I, or J) and the solvent accessibility (SA) cut-off employed.

FIG. 3 shows a schematic diagram of the open reading frame of plasmid pTG3. An antigen of interest is expressed in fusion with a conserved DYKDDDDK-tag, a KDIR trypsin site, and M13 bacteriophage gIII.

FIG. 4 shows 3D structural models of human IL8 with predictions of the amino acids of the epitope of antibody AbIL8-5 indicated based on the method described in Example 3. The indicated amino acids have been assigned a ranking by Seqitope. The lower the ranking the higher the likelihood of the amino acid being in the epitope. Amino acid position with rank 1-4 (black), 5-8 (grey), and 9-12 (white). (A) Prediction with no SA value cut-off; (B) Prediction using an SA value cut-off of >23; (C) Prediction using DYKDDDDK normalisation and no SA value cut-off.

FIG. 5 shows antibody binding to wildtype IL8 and IL8-mHKF (containing the mutations H18A, K20A and F21A) in an ELISA assay. AbIL8-3 and AbIL8-5 bind IL8-mHKF significantly lower than wildtype IL8 which is in accordance with the epitopes determined by Seqitope. The top three residues important for binding of AbIL8-5 is K20, H18 and F21, while H18 and F21 are in the top three most important for binding of AbIL8-3 (see Table 9).

FIG. 6 shows 3D structural models of PTXa with predictions of the amino acids of the epitope of the 1B7 antibody indicated based on the method described in Example 4. The indicated amino acids have been assigned a ranking by Seqitope. The lower the ranking the higher the likelihood of the amino acid being in the epitope. Amino acid position with rank 1-4 (black), 5-8 (grey), and 9-12 (white). (A) Prediction with no SA value cut-off; (B) Prediction using an SA value cut-off of >23; (C) Prediction using AbPTX-1 antibody normalisation and no SA value cut-off; (D) Indication of the published epitope of the 1B7 antibody.

FIG. 7 shows 3D structural models of SARS-COV2-RBD with predictions of the amino acids of the epitope of the Ty1 antibody indicated based on the method described in Example 5. The indicated amino acids have been assigned a ranking by Seqitope. The lower the ranking the higher the likelihood of the amino acid being in the epitope. Amino acid positions with rank 1-4 (black), 5-8 (grey), and 9-12 (white). (A) Prediction without any SA value cut-off; (B) Prediction using an SA value cut-off of >23; (C) Prediction using AbRBD-1 antibody normalisation and no SA value cut-off; (D) Indication of the published epitope of the Ty1 antibody in Hanke et al, 2020.

FIG. 8 shows a 3D structural model of SARS-COV2-RBD indicating the top and bottom six amino acid residues as ranked by the Seqitope method described in Example 5. The top six ranked amino acid residues are shown in black, and the bottom six ranked amino acids are shown in grey.

FIG. 9 shows SDS-PAGE gel of purified SARS-COV2-RBD proteins as described in Example 6. The gel shows SeeBlue Plus2 prestained ladder (lane 1), 1 ug RBD-wt (lane 2), 1 ug RBD-R (lane 3) and 1 ug RBD-YQ (lane 4).

FIG. 10 shows sensorgrams from the SPR analysis described in Example 6, with corresponding numbers presented in Table 13.

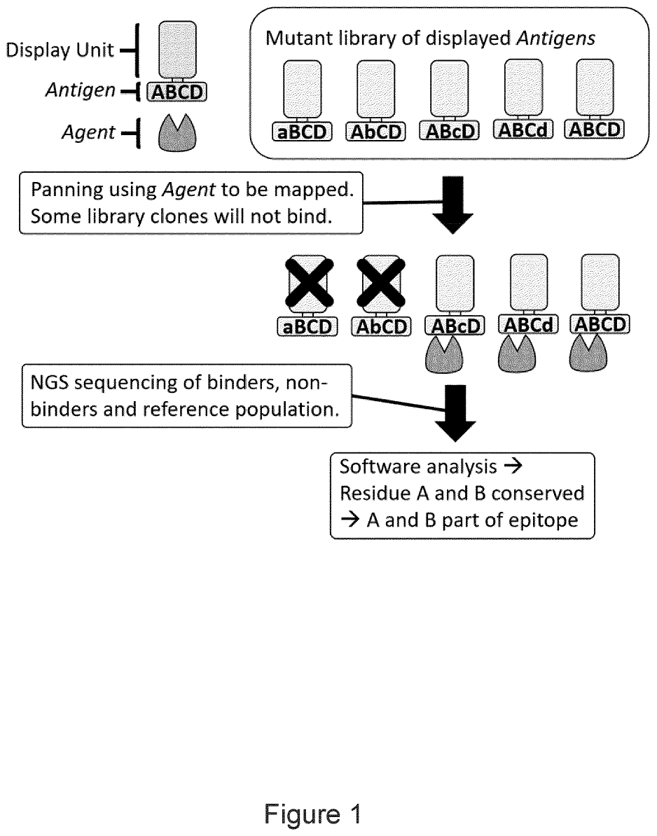

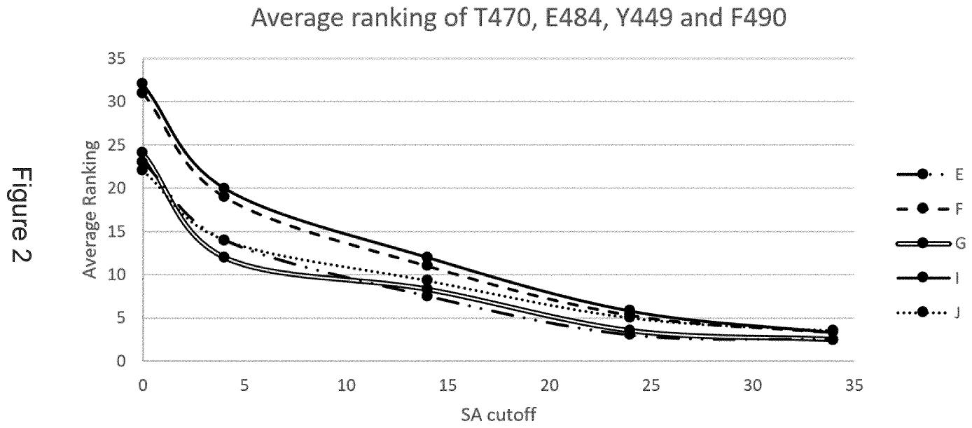

### EXAMPLES

### Overview

The method of the present invention (hereafter referred to as “Seqitope”) is a method for precise antibody epitope mapping of complex epitopes, combining high-resolution output with high-throughput capacity. Seqitope randomly introduces mutations to an antigen to be screened and displays the mutated antigen in a mixed library, omitting the need for purified antigen. Each amino acid position may be mutated to any of the twenty natural amino acids, thereby providing large antigen libraries to be sampled by an agent of interest (e.g. an antibody). Seqitope then compares the amino acid diversity (measured using Shannon entropy) for each amino acid position in a reference library versus the experimental post-panning library, with the final Seqitope output being a ranking of all antigen amino acid positions according to their likelihood of contributing to the epitope. This approach leads to the delivery of epitope mapping having single amino acid resolution of linear as well as conformational epitopes at a high-throughput scale.

### Materials and Methods

The materials and methods described below relate to the experiments carried out in Examples 1 to 5.

### Production of Antigen Phage Library

A 50 ul GeneMorph II Random Mutagenesis (Agilent) reaction was set up according to the manufacturer's instructions to reach a rate of 2-3 nucleotide mutations per antigen library clone. Three antigen sequences were amplified by PCR using the templates pPTX-G, pTwist-EF1alpha nCov-2019-S-2×Strep, and cDNA MGC: 9211 respectively. Those sequences were:
Pertussis Toxin subunit A (PTXa);
SARS-COV2-RBD (RBD); and,
Human IL8.

PCR amplification was carried out using the primer pairs shown in Table 1 below.

TABLE 1
Primer pairs used for PCR amplification of
antigen sequences. Restriction sites for
use in subsequent molecular cloning are
underlined (ACCGGT = AgeI; GCGGCCG = NotI).

| Antigen | Primer pair |
|---|---|
| PTXa | 5′-TATACTACCGGTGACGATCCTCCCGC |
| | CACCGTATAC-3′ |
| | 5′-ATGTATGCGGCCGCTTCGGCCTGCCG |
| | CGCCATGCAAG-3′ |
| RBD | 5′-ATACAACCGGTAGGGTACAACCTACG |
| | GAATCTATCG-3′ |
| | 5′-TACATGCGGCCGCAAAATTGACGCAT |
| | TTATTCTTCAC-3′ |
| IL8 | 5′-ATACAACCGGTAGTGCTAAAGAACTT |
| | AGATGTC-3′ |
| | 5′-TACATGCGGCCGCTGAATTCTCAGCC |
| | CTCTTCAAAAACTTC-3′ |

The PCR products were gel purified and digested using AgeI-HF+NotI-HF (NEB). The digested PCR products were ligated into similarly digested plasmid pTG3 using T4 DNA ligase (NEB) according to manufacturer's instructions. The ligation products were electroporated into XL1-blue electrocompetent cells (Agilent) according to manufacturer's instructions. The final phage libraries were produced using standard protocols (Carlos F Barbas III, Dennis R. Burton, Gregg J. Silverman. 2001. Phage Display: A Laboratory Manual 1st Edition (ISBN-13:978-0879697402). CSHL Press) and had final titers of 1×10⁷-1×10⁸ cfu/mL.

### Epitope Panning

1 μg of the antibody to be tested was mixed with 10-60 μL of the antigen phage library in a total volume of 400 μL of PBS-Tween with 1% Bovine Serum Albumin (BSA). That mixture was incubated rotating at 4° C. overnight. 10 μL washed Protein G magnetic beads (NEB) was added to each antibody-phage library mix, followed by an incubation at room temperature with rotation for 1 hr. The beads were pelleted using a magnetic stand and the non-bound phages transferred to a new tube.

The beads were washed four times in PBS-Tween using a magnetic stand. 50 μL of 0.25% trypsin (Gibco™ Trypsin #15090-046 Thermo Fisher) diluted in water was added, followed by an incubation at room temperature with rotation for 1 hr.

A magnetic stand was used to transfer the supernatants containing the trypsin eluted phages to new microtubes. The remaining beads were resuspended in 25 μL of 0.2 mg/ml Aprotinin (1 mg/mL, #A2132, AppliChem) diluted in water. The beads were pelleted using a magnetic stand and the Aprotinin supernatants were pooled with the trypsin eluted phages.

### Sequencing of Eluted Clones

The eluted phage library clones, the non-bound clones, and the pre-panning phage libraries were amplified using PCR. Primers with the appropriate Illumina handles were used (see Table 2). Due to limitations in Illumina-reading length, the two larger antigens RBD and PTXa were split into two reactions (i.e. one reaction covering the 5′ end of the antigen and one reaction covering the 3′ end including a central overlap).

20 μL KAPA HiFi HotStart ReadyMix (KAPA Biosystems) reactions were set up for each sample according to the manufacturer's instructions. An annealing temperature of 68° C. and an elongation time of 15 secs was used. For the experimental reactions, 5 μL eluted phage sample was used as a template. For the control reactions, 0.1 μL of the non-bound clones and the pre-panning phage library samples were used as a template. Samples were taken from each KAPA PCR reaction after 5, 10, 15, and 20 PCR cycles and analysed by gel electrophoresis. Based on the band intensity of the PCR products at different cycle numbers, an optimal cycle number to reach a total yield of 50-100 ng of PCR product was determined for each reaction.

New KAPA reactions using the optimal cycle number were performed for each sample type to create the PCR1 products (i.e. eluted phage sample, non-bound phage sample, and pre-panning phage library sample).

PCR1 samples were purified using 21 μl of MagSI NGSPrep Plus purification beads (TATAA, MDKT00010075) following the supplier's instructions and samples were eluted in 12 μl of Elution Buffer (Qiagen, 19086).

6 μl of the PCR1 eluate was subjected to amplification in a second PCR (PCR2) with the following reaction setup: 10 μl KAPA HiFi Hotstart 2× Master Mix, 1 μl of 10 UM i7 index primer and 1 μl of 10 μM i5 index primer (see Table 2 and Hugerth et al. 2014. Appl Environ. Microbiol., 80(16):5116-23). Nuclease free water was added to a reaction size of 20 μl.

The PCR conditions for PCR2 were as follows: 98° C. for 2 mins; 8 cycles of 98° C. for 20 secs, 62° C. for 20 secs, 72° C. for 15 secs and a final elongation at 72° C. for 2 mins. PCR2 samples were purified using 20 μl of MagSI NGSPrep Plus purification beads.

TABLE 2
PCR primers used for amplification of PTXa-1,
RBD-1, and IL-8 for sequencing applications.

| Primer | Sequence |
|---|---|
| PT_fp1 | 5′-ACACTCTTTCCCTACACGACGCTCTTC |
| | CGATCTACCGTGGCCCAGGCGACCGGT-3′ |
| PT_rp2 | 5′-AGACGTGTGCTCTTCCGATCTCCACCA |
| | CCCTCTGCGGCCGC-3 |
| PT_rp1 | 5′-AGACGTGTGCTCTTCCGATCTGTGTGC |
| | CAGATATCCGCTCTG-3′ |
| PT_fp2 | 5′-ACACTCTTTCCCTACACGACGCTCTTC |
| | CGATCTCGAACGCGCCGGCAGGGGCAC-3′ |
| RBD_rp1 | 5′-AGACGTGTGCTCTTCCGATCTGTAAAG |
| | GTAGTTATAATTTCC-3′ |
| RBD_fp2 | 5′-ACACTCTTTCCCTACACGACGCTCTTC |
| | CGATCTGCAAATTGCGCCAGGGCAGAC-3′ |
| Index | Described in Hugerth et al, 2014. Appl |
| Primers | Environ Microbiol., 80(16: 5116-5123 |

Library quality was determined for all samples by concentration measurements using Quant-IT dsDNA Assay Kit, High sensitivity (ThermoFisher, Q33120) and analysis of ten samples and a negative control on a Bioanalyzer DNA HS chip. The PCR2 samples were pooled in equal molar amounts to create the final library for Illumina MiSeq sequencing. The final pooled library was run on an Illumina MiSeq using the v3 kit with a 2×300 bp read setup.

### Bioinformatic Analysis

A bioinformatics pipeline was built in Snakemake (Köster et al, 2012. Bioinformatics, 28(19): 2520-2522) that performs the following steps:
Quality-filtering of read-pairs using Cutadapt (Martin, 2011. EMBnet J., 17(1)). This step removes low quality bases in the 3′ ends of reads, discards read-pairs lacking the expected primer sequences in the 5′ ends, removes primer sequences from 5′ ends, discards read-pairs that were too short after trimming, and discards read-pairs including Illumina adapters.
Alignment of filtered read pairs to the reference antigen DNA sequence using Bowtie2 (Langmead et al, 2012. Nat. Methods, 9:357-359). Conversion of the output of Bowtie2 to BAM files, and sorting, indexing, and merging of these with SAMtools (Li et al, 2009. Bioinformatics, 25(16): 2078-2079).
Removing aligned reads from the BAM files that have insertions/deletions and/or more than a certain number of mismatches relative to the antigen sequence, using the custom script filter_reads.py that utilises the pysam package.
Obtaining counts of each of the four nucleotides at each position of the antigen sequence based on the aligned reads. Only reads fulfilling a cut-off on mapping quality and bases fulfilling a cut-off on sequence quality, were used through use of the custom script count_nucleotides.py.
Converting counts of nucleotides into counts of amino acids for each position of the antigen protein sequence, using the custom script count_amino_acids.py.
Extracting solvent accessibility for each amino acid of the antigen based on the structure of the protein in Protein Data Bank (PDB) (Berman et al, 2003. Nat Struct Mol. Biol., 10:980) and using the custom script extract_rsa that utilises the DSSP software (Kabsch and Sander, 1983. Biopolymers, 22:2577-2637).
In case no PDB entry is available, predicting the solvent accessibility of each amino acid using the custom script predict_rsa that utilises rawMSA (Mirabello and Wallner, 2019. PLOS One, 14(8):e0220182). Calculating Shannon entropy at each amino acid position based on the amino acid frequencies and predicting the epitope based on the ratio of Shannon entropy in the antibody bound library versus a reference library using the custom script predict_epitope.py. The reference library used is either the pre-panning library (i.e. before antibody binding) or a control panning (e.g. anti-DYKDDDDK).

### Phage Displaying the Alanine Mutant IL8-mHKF

Overlapping PCR was used to create an alanine mutant of IL8 using standard PCR protocols. In short, the “lac_fwd” and “IL8-rp (H-KF_ala.mut)” primers (see Table 3) were used to amplify the 5′ end of IL8 and the “IL8-fp (H-KF_ala.mut)” and “IL8_rev/NotI” primers (see Table 3) were used to amplify the 3′ end of IL8. Purified 5′ and 3′ PCR fragments were then amplified using the “IL8_72/AgeI” and “IL8_rev/NotI” primers (see Table 3) to create the full length mutated IL8 gene containing the mutations H18A, K20A, and F21A. The PCR product was cloned into the vector pTG3 using AgeI and NotI restriction sites and standard ligation protocols. Phage displaying IL8-mHKF was produced and purified as previously described herein.

TABLE 3
Primer pairs used for introduction of H18A,
K20A, and F21A mutations in IL8. Lower
case letters in the primer IL8-fp(H-KF_ala.mut)
indicate introduced mutations. Restriction
sites for use in subsequent molecular
cloning are underlined
(ACCGGT = AgeI; GCGGCCG = NotI).

| Primer | Sequence |
|---|---|
| Lac_fwd | 5′-AATGTGAGTTAGCTCACTCA |
| | TTAGG-3′ |
| IL8-rp | 5′-CTTTGATAgcTgcGGGGgcG |
| (H-KF_ala.mut) | AAAGGTTTGGAGTATGTCTTTAT |
| | GC-3′ |
| IL8-fp | 5′-CCTTTCgcCCCCgcAgcTAT |
| (H-KF_ala.mut) | CAAAGAACTGAGAGTGATTGAGA |
| | G-3′ |
| IL8_rev/NotI | 5′-TACATGCGGCCGCTGAATTC |
| | TCAGCCCTCTTCAAAAACTTC-3′ |
| IL8_72/AgeI | 5′-ATACAACCGGTAGTGCTAAA |
| | GAACTTAGATGTC-3′ |

### IL8 ELISA

ELISA plates (442404, Nunc) were coated using AbIL8-1, AbIL8-2, AbIL8-3, AbIL8-4, AbIL8-5, anti-M13 (27-9420-01, Amersham Pharmacia) or anti-DYKDDDDK (MA1-91878, invitrogen) antibodies at 5 μg/mL in 0.05M NaCO₃, pH 9.6. The plates were incubated overnight at 4° C. Following that incubation, the plates were washed twice with PBS-Tween and blocked using 100 μl of PBS+1% BSA at room temperature for 1 hr. The plates were then washed twice with PBS-Tween.

A dilution series of phage displaying either wildtype IL8 or the alanine mutant IL8-mHKF was added to the plates. After incubation at room temperature for 2 hrs, the plates were washed four times in PBS-Tween. 50 μl of anti-M13-HRP (11973-MM05T-H, Sino Biological) diluted 1:5000 in PBS-Tween was added to each well of the plates. After incubation at room temperature for 2 hrs, the plates were washed four times in PBS-Tween. 50 μl/well 1-Step™ Ultra TMB-ELISA Substrate Solution (ThermoFisher Scientific) was added to each well. After 5 mins of development time the reactions were stopped by addition of 50 μl of 2M H₂SO₄. The absorbance in each well were measured at 450 nm using a spectrophotometer.

### Create RBD Mutants

Overlapping PCR was used to introduce the mutations Q493R and the combination mutations N450Y+L455Q into RBD using standard PCR protocols. In short, the “RBD-AfIII-AgeI” and “R-RBD rp” primers (see Table 4) were used to amplify the 5′ end of RBD and the “R-RBD fp” and “RBD2/NotI rp” primers (see Table 4) were used to amplify the 3′ end of RBD. Purified 5′ and 3′ PCR fragments were then amplified using the “RBD-AfIII-AgeI” and “RBD2/NotI rp” primers (see Table 4) to create the full length mutated RBD gene containing the mutation Q493R. The final PCR product called RBD-R was cloned into the vector pES (a modified version of the pcDNA3 vector which encodes a C-terminal StrepTag II) using AfIII and NotI restriction sites and standard ligation protocols resulting in the mammalian expression plasmid PES-RBD-R.

The same procedure using “YQ”-primers (see Table 4) was used to create the N450Y+L455Q mutant resulting in the mammalian expression plasmid pES-RBD-YQ.

TABLE 4
Primers used for introduction of Q493R and
N450Y + L455Q mutations in RBD. Lower
case letters in the primer indicate
introduced mutations. Restriction sites
for use in subsequent molecular cloning
are underlined
(CTTAAG = AfIII; GCGGCCG = NotI).

| Primer | Sequence |
|---|---|
| RBD-AfIII/ | 5′-ATAGACTCTTAAGtACCGGT |
| AgeI | AGGGTACAACCTACGG-3′ |
| RBD2/NotI rp | 5′-TACATGCGGCCGCTTTATTC |
| | TTCACCAGATTAGTAG-3′ |
| R-RBD fp | 5′-GTTATTTCCCATTGcgaTCC |
| | TATGGTTTTCAACCGACG-3′ |
| R-RBD rp | 5-GTTGAAAACCATAGGAtcgCA |
| | ATGGGAAATAACAATTGAATCC-3′ |
| YQ-RBD fp | 5′-GGAAATTATtacTACCTTTA |
| | CAGGcagTTCCGGAAGAGTAATC |
| | TGAAGC-3′ |
| YQ-RBD rp | 5′-CTCTTCCGGAActgCCTGTA |
| | AAGGTAgtaATAATTTCCCCCTA |
| | CCTTTGAGTC-3′ |

### Production and Purification of RBD Mutants

The plasmids pES-RBD-wt, pES-RBD-R and pES-RBD-YQ were propagated in XL1 Blue bacteria and midi prepped (Thermo Scientific GeneJET Plasmid Midiprep Kit). The plasmids were transformed into Expi293F cells (ThermoFisher Scientific) according to manufacturer's recommendations. The cells were harvested after 7 days and centrifuged. The supernatants were transferred to new tubes. DNAse (New England Biolabs) and Biolock (IBA Lifesciences) was added to the supernatants to digest any free chromosomal DNA and bind up any free biotin. After overnight incubation at +4 degrees Celsius the supernatants were spun once again followed by a 0.45 μm filtration. The cleared supernatants were purified using a StrepTrap XT (Cytiva) column using the recommended protocols. The proteins were eluted using 50 mM Biotin in PBS pH 8, then immediately buffer changed to PBS using a PD-10 column (Cytiva).

SDS Page 1 ug of each purified RBD protein (RBD-wt, RBD-R, RBD-YQ) were run on a 10% Bis-Tris NuPAGE gel (Thermo Fisher Scientific) using LDS buffer and reducing agent according to manufacturer's standard protocol. The gel was stained using SimplyBlue SafeStain (Thermo Fisher Scientific) using the “fast protocol” according to the manufacturer's recommendations.

### Surface Plasmon Resonance Analysis

Surface plasmon resonance (SPR) analysis was performed on a Biacore 8k using a CM5 series S chip (GE Healthcare #BR1005-30), with anti-human Fc capture kit (Cytiva #292346000). HBS-EP+ (Cytiva #BR100826) was used as running buffer and 10 mM glycin-HCl, pH 2.1 (GE Cytiva) was used as regeneration buffer. Each IgG and Ty1-Fc was diluted to 3 ug/ml in running buffer and captured at a flow of 10 ul/min for 120s which resulted in a capture level at approx. 100-250 RU (up to 400 RU). Following capture of the antibodies, five concentrations (0.16, 0.8, 4, 20, 100 nM) of RBD protein was injected at a flow of 30 ul/min, association 120s, dissociation 180s. After the dissociation phase, the surface was regenerated using two pulses of 10 nM Glycine-HCl, pH 2.1, and the system was allowed to stabilize for 600 s before the next capture. For each antibody investigated, a blank cycle was also performed, where running buffer was injected as analyte instead of RBD protein and used for background subtraction before fitting data for each antibody to a 1:1 Langmuir binding model.

### Example 1

Seqitope was used to map the epitope of the monoclonal antibody 1B7 which targets the Pertussis Toxin subunit A (PTXa) antigen. Monoclonal antibody 1B7 has a published epitope (Sutherland and Maynard, 2009. Biochemistry, 48(50): 11982-11993) and so can be used to assess the validity of the Seqitope output.

In order to determine a suitable antigen mutation ratio, two PTXa libraries were created. In the first library, an average of 2.2 nucleotide mutations were introduced per library member. In the second library, an average of 6.5 nucleotide mutations were introduced per library member.

The two libraries were used in two different concentrations during the epitope panning experiments: either 1:20 or 1:1 of the library in a total volume of 400 μL PBS-Tween with 1% BSA including 1 μg of the antibody to be mapped. A second round of panning was also carried out to assess whether this would improve the results (see Table 5: Protocol A-R2). The different experimental protocols are summarised in Table 5.

The Seqitope software was used to rank all amino acid residues in the antigen according to their likelihood of being part of the epitope. In short, this ranking was done by comparing the amino acid diversity (measured using Shannon entropy) for each amino acid position in the pre-panning library vs. the post-panning library. The average ranking of the reported epitope residues recognised by the 1B7 antibody (i.e. R79, H83, Y148, N150) was then used as a metric to assess which protocol yielded the best results. It is important to note that the full epitope recognised by the 1B7 antibody may well include other amino acid positions not investigated in the published paper (Sutherland and Maynard, 2009. Biochemistry, 48(50): 11982-11993).

To further refine the output obtained, the data was filtered using different SA value cut-offs. Residues with a low solvent accessibility are unlikely to interact with an antibody since they are buried deep within the core of the antigen protein. The lower the SA value for an amino acid, the more likely it is to be inaccessible to an antibody. Three different SA value cut-offs were used: SA value >3 cut-off (i.e. amino acids with SA values between 0-3 filtered); SA value >13 cut-off (i.e. amino acids with SA values between 0-13 filtered); and, SA value >23 cut-off (i.e. amino acids with SA values between 0-23 filtered) (see Table 5). Increasing the SA value cut-off further would exclude Y148 which has an SA value of 24, thereby resulting in a false negative.

Results are presented in terms of the average ranking of the amino acids identified as being in the epitope recognised by the 1B7 antibody in Sutherland and Maynard, 2009 (i.e. R79, H83, Y148 and N150). The lower the average ranking the better agreement with the published epitope.

The best results for a single panning experiment were obtained with Protocol D, which uses the lower mutation rate of 2.2 nucleotides per library member, an SA value cut-off of >23, and a more dilute antigen phage library (1:40). It was also observed that adding a second round of panning led to an improved result (see Table 5: compare Protocols A and A-R2).

TABLE 5
Comparison of protocols using different library mutation
rate and reference libraries. The average rankings of
the amino acids reported to be recognised by the 1B7
antibody (i.e. R79, H83, Y148 and N150) are listed.

| Protocol | A | B | C | D | A-R2 |
|---|---|---|---|---|---|
| Average nucleotide mutation rate | 6.5 | 6.5 | 2.2 | 2.2 | 6.5 |
| per library member | | | | | |
| PTXa library ratio | 1:1 | 1:40 | 1:1 | 1:40 | 1:1 |
| Average rank (no SA cut-off) | 19.0 | 15.0 | 13.0 | 12.0 | 12.0 |
| Average rank (SA >3 cut-off) | 19.0 | 15.0 | 13.0 | 12.0 | 12.0 |
| Average rank (SA >13 cut-off) | 13.0 | 10.0 | 8.0 | 7.3 | 8.3 |
| Average rank (SA >23 cut-off) | 7.0 | 5.8 | 5.8 | 5.5 | 5.0 |

### Example 2

Seqitope was used to map the epitope of the nanobody Ty1 which targets the antigen SARS-COV2-RBD. The Nanobody Ty1 has a previously published epitope (Hanke et al, 2020. Nat. Commun., 11, 4420), and so can be used to assess the validity of the Seqitope output.

Three different antigen library concentrations were used during the epitope panning experiments: 1:2; 1:6; and, 1:100. Those libraries each had an average of 2.8 nucleotide mutations per library member. The different experimental protocols used in each are summarised in Table 6.

TABLE 6
Comparison of protocols using different library concentrations and reference
libraries. The average rankings of the amino acids reported to be recognised
by the Ty1 nanobody (i.e. T470, E484, Y449 and F490) are listed.

| Protocol | E | F | G | I | J |
|---|---|---|---|---|---|
| RBD library ratio | 1:2 | 1:6 | 1:100 | 1:6 | 1:100 |
| Reference library | Pre-panning | Pre-panning | Pre-panning | Non-bound | Non-bound |
| Average rank (no SA cut-off) | 23.0 | 31.0 | 24.0 | 32.0 | 22.0 |
| Average rank (SA >3 cut-off) | 14.0 | 19.0 | 12.0 | 20.0 | 14.0 |
| Average rank (SA >13 cut-off) | 7.5 | 11.0 | 8.3 | 12 | 9.3 |
| Average rank (SA >23 cut-off) | 3.0 | 5.3 | 3.5 | 5.8 | 5.0 |
| Average rank (SA >33 cut-off) | 2.5 | 3.3 | 2.5 | 3.3 | 3.5 |

The Seqitope software used either the pre-panning library or the non-bound fraction as a reference to the post-panning library to determine the ranking of amino acid residues. The average ranking of a subset of the amino acid residues reported to be recognised by the Ty1 nanobody (T470, E484, Y449 and F490) was then used as a metric to assess which protocol yielded the best results.

The data were filtered using different SA value cut-offs. Four different SA value cut-offs were used: SA>3 (i.e. amino acids with SA values between 0-3 filtered), SA>13 (i.e. amino acids with SA values between 0-13 filtered), SA>23 (i.e. amino acids with SA values between 0-23 filtered) and SA>33 (i.e. amino acids with SA values between 0-33 filtered). The best overall results were obtained using Protocol E, which used a 1:2 dilution of the phage library in combination with using the pre-panning library as a reference (see Table 6). Using Protocol E and an SA value cut-off of >33 resulted in T470, E484, Y449 and F490 being ranked in the top four positions (average rank=2.5). The use of the non-bound fraction as a reference instead of the pre panning library did not significantly improve the results (see Table 6: compare Protocols F and I or Protocols G and J).

### Example 3

Certain amino acid positions in the antigen may be critical to its overall structure and stability. Accordingly, mutagenesis of such positions may result in a false high ranking in the Seqitope output. Using SA cut-offs is one way of handling this problem (i.e. by excluding amino acids that should be inaccessible to an antibody). An additional approach is to include a control panning experiment measuring the background signal from each amino acid position.

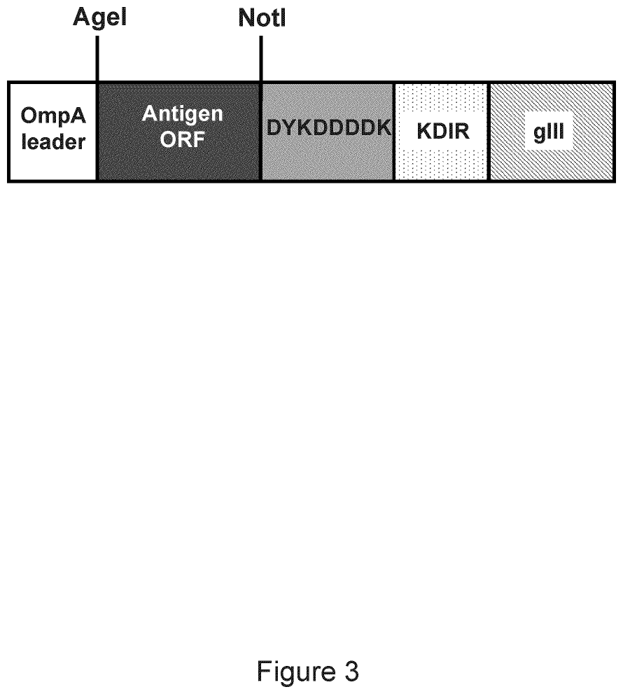

In this Example, the antigen was expressed as a fusion protein with a DYKDDDDK-tag (see FIG. 3) that is not subjected to mutagenesis and will therefore be fully conserved in all library members. In that way, any signal from antigen amino acid positions in a DYKDDDDK targeting control panning are caused by structural, stability, or expression issues in the antigen and can be used to normalise the epitope mapping results.

Seqitope was used to map the epitope of five monoclonal antibodies targeting human IL8. In parallel, DYKDDDDK control panning was also carried out on the same antigen libraries. None of the five antibodies used had a known epitope.

An antigen library concentration of 1:6 was used during the epitope panning experiments and the libraries each had an average of 2.2 nucleotide mutations per library member. The Seqitope software used the pre-panning library as a reference to the post-panning library to determine the ranking of amino acid residues. The different experimental protocols used in each are summarised in Table 7.

In order to quantify the contribution of the DYKDDDDK-normalisation, the top eight residues predicted by Seqitope were investigated for each of the five antibodies. A total of 26 of 40 predicted epitope residues were identical irrespective of whether DYKDDDDK normalisation was used (see Table 7).

TABLE 7
Comparison of the average SA value of the non-identical predicted
epitope residues (numbers of residues given in parentheses) when
using no normalisation or using DYKDDDDK normalisation.

| | AbIL8-1 | AbIL8-2 | AbIL8-3 | AbIL8-4 | AbIL8-5 |
|---|---|---|---|---|---|
| Monoclonal antibody | antibody | antibody | antibody | antibody | antibody |
| No normalisation | 36 (6) | 11 (2) | 15 (2) | 24 (3) | 29 (2) |
| DYKDDDDK normalisation | 38 (6) | 79 (2) | 62 (2) | 28 (3) | 67 (2) |

When the average SA values for the non-identical residues were considered, the DYKDDDDK normalised dataset had higher SA values. That observation indicated that the DYKDDDDK normalised dataset contains more exposed amino acid residues (i.e. amino acids that more likely to be naturally accessible to an antibody and less likely to be ranked highly due to their involvement in structure and stability of the antigen).

Table 8 presents the top twelve epitope residues predicted by Seqitope for the AbIL8-5 antibody using the following experimental set-ups:
1) Protocol F, no normalisation, no SA cut-off;
2) Protocol F, no normalisation, SA cut-off of >23; and,
3) Protocol F, DYKDDDDK normalisation, no SA cut-off.

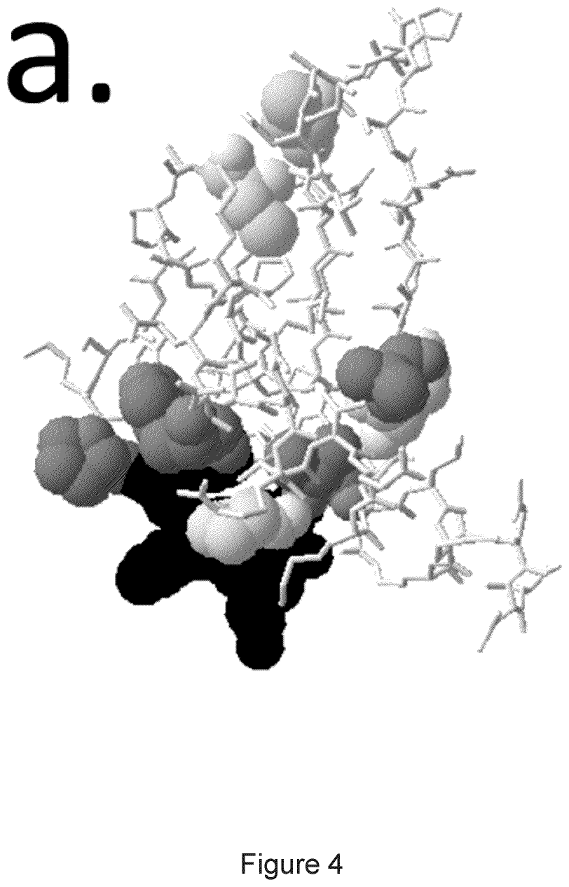

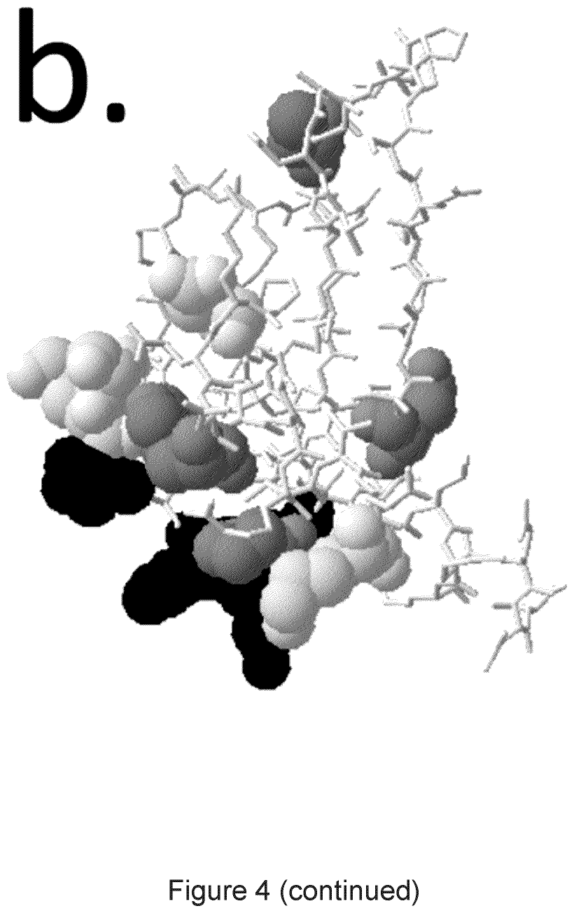

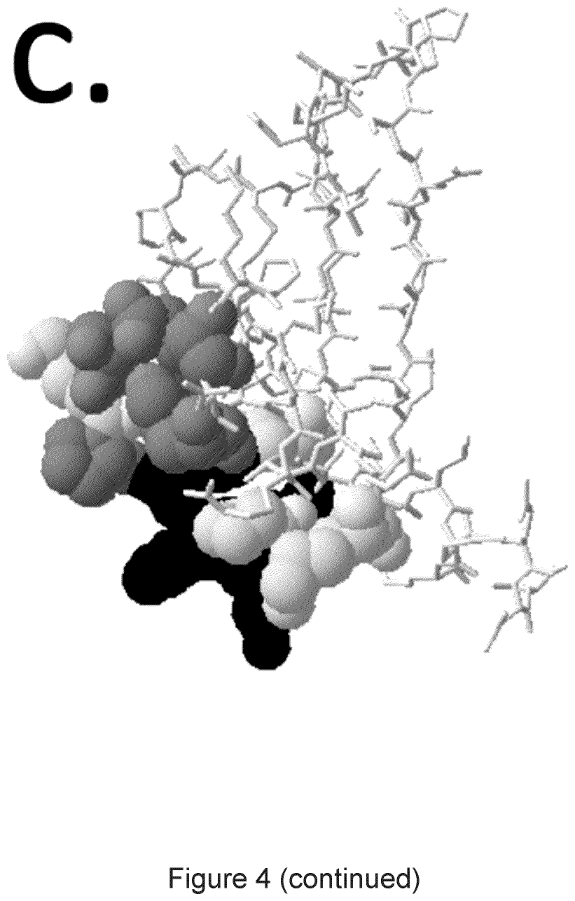

The positioning of those amino acids in the structure of the human IL8 antigen are shown in FIG. 4. From a steric and/or distance perspectives, some of the predicted amino acids are unlikely to be part of the epitope (i.e. considered to be false positives). Notably, when DYKDDDDK normalisation is applied (see FIG. 4C), the amino acid residues predicted to form part of the epitope are much more “condensed” on IL8 and there are no obviously false positives in other areas of the molecule (see FIGS. 4A and 4B).

TABLE 8
The top twelve hIL8 amino acid residues bound by AbIL8-5
according to Seqitope when different SA cut-offs and/or
normalisation is applied. Amino acid residues considered
unlikely to form part of the epitope from a steric and/or
distance perspective are written in parentheses.

| | | Protocol F, |
|---|---|---|
| Protocol F, | Protocol F, | DYKDDDDK normalisation, |
| No SA cut-off | SA cut-off >23 | no SA cut-off |
| K20 | K20 | K20 |
| H18 | H18 | H18 |
| F21 | F21 | F21 |
| F17 | P16 | F17 |
| P16 | W57 | P16 |
| (L25) | (E24) | S14 |
| W57 | P19 | E55 |
| I22 | (C7) | W57 |
| (E24) | K64 | K64 |
| P19 | K15 | L43 |
| (C7( | S14 | P19 |
| (C9) | (D52) | K15 |
| Positions considered | Positions considered | Positions considered |
| unlikely = 4 | unlikely = 3 | unlikely = 0 |

To verify the AbIL8-5 epitope determined by Seqitope, an ELISA assay was performed. Binding of the wildtype hIL8 and the alanine mutant hIL8-mHKF (containing the mutations H18A, K20A, and F21A) was investigated for the antibodies AbIL8-1, AbIL8-2, AbIL8-3, AbIL8-4, AbIL8-5, anti-DYKDDDDK, and anti-M13 antibodies.

TABLE 9
The top five residues important for antibody binding according
to Seqitope. Residues mutated in IL8-mHKF are underlined.

| AbIL8-1 | AbIL8-2 | AbIL8-3 | AbIL8-4 | AbIL8-5 |
|---|---|---|---|---|
| L66 | C34 | H18 | E29 | K20 |
| E29 | C50 | Y13 | P32 | H18 |
| V58 | C7 | F21 | I39 | F21 |
| E70 | R6 | R47 | S30 | F17 |
| F65 | C9 | K15 | T37 | P16 |

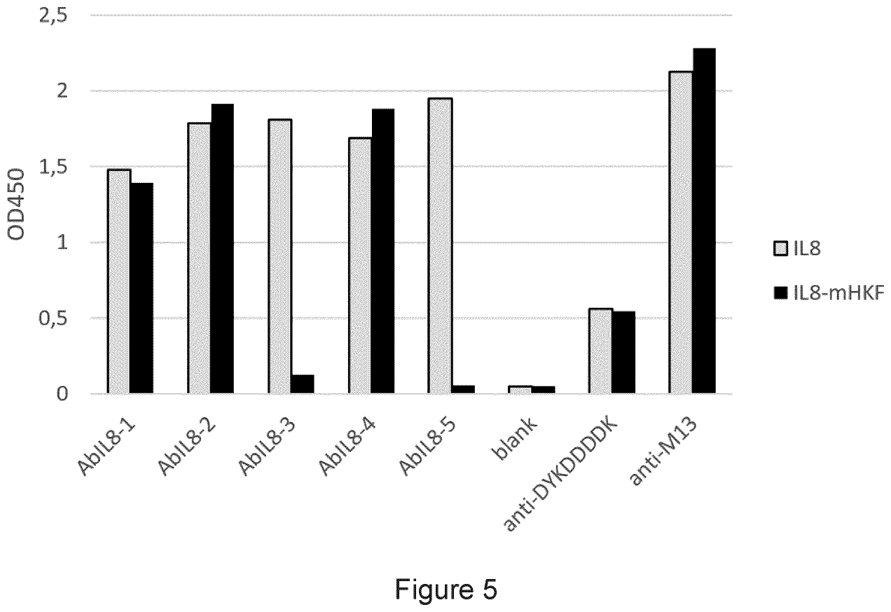

No significant difference in binding between IL8-wt and IL8-mHKF was observed other than for AbIL8-5 and AbIL8-3 (see FIG. 5). Those results were in accordance with the epitopes determined by Seqitope since the top three residues important for binding of AbIL8-5 were determined as K20, H18 and F21, while H18 and F21 were in the top three most important for binding of AbIL8-3 (see Table 9).

### Example 4

In contrast to Example 3, removal of false positives from the PTXa dataset did not involve the use of a DYKDDDDK control panning experiment. However, the same methodology can be applied using another antibody mapped in parallel to the 1B7 monoclonal antibody. The antibody AbPTX-1 also binds PTXa but has an epitope that is distinct from the one recognised by 1B7, which is important for the success of this approach.

Seqitope data from AbPTX-1 was used to normalise the 1B7 data presented in Example 1. The protocol for the normalisation is as described for the DYKDDDDK control panning normalisation carried out in Example 3.

Table 10 presents the top twelve epitope residues predicted by Seqitope for the 1B7 antibody using:
1) Protocol D, no normalisation, no SA cut-off;
2) Protocol D, no normalisation, SA cut-off of >23; and,
3) Protocol D, AbPTX-1 normalisation, no SA cut-off.

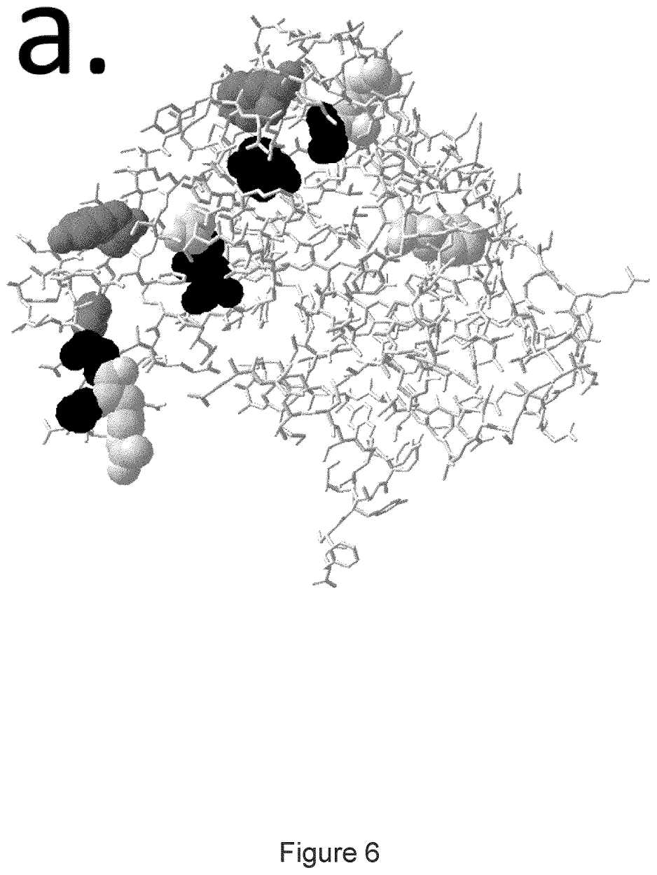

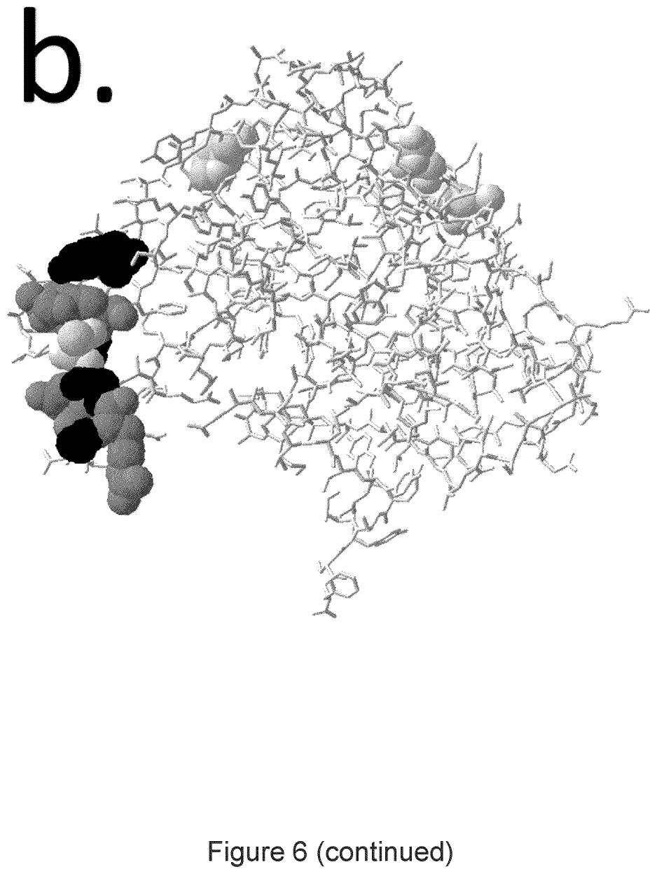

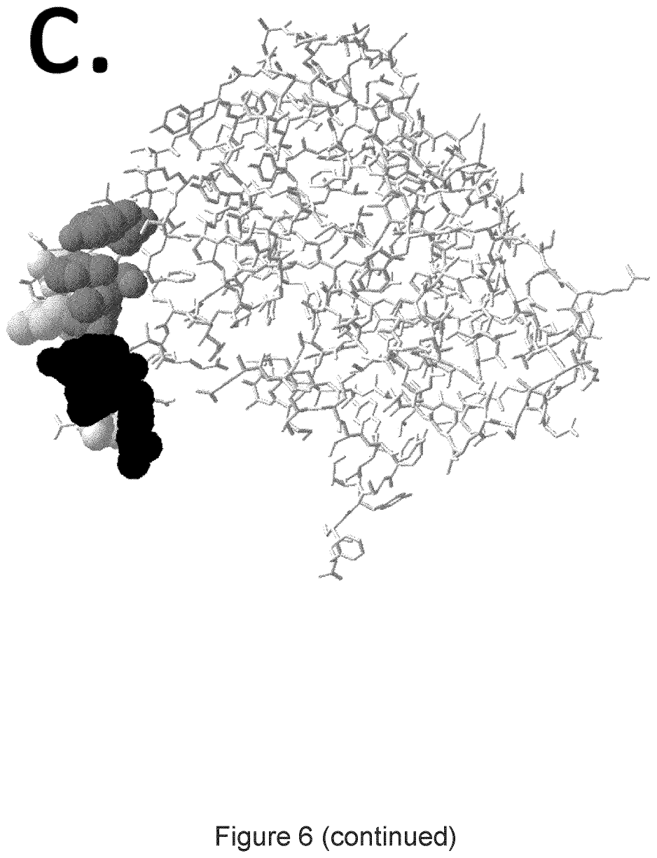

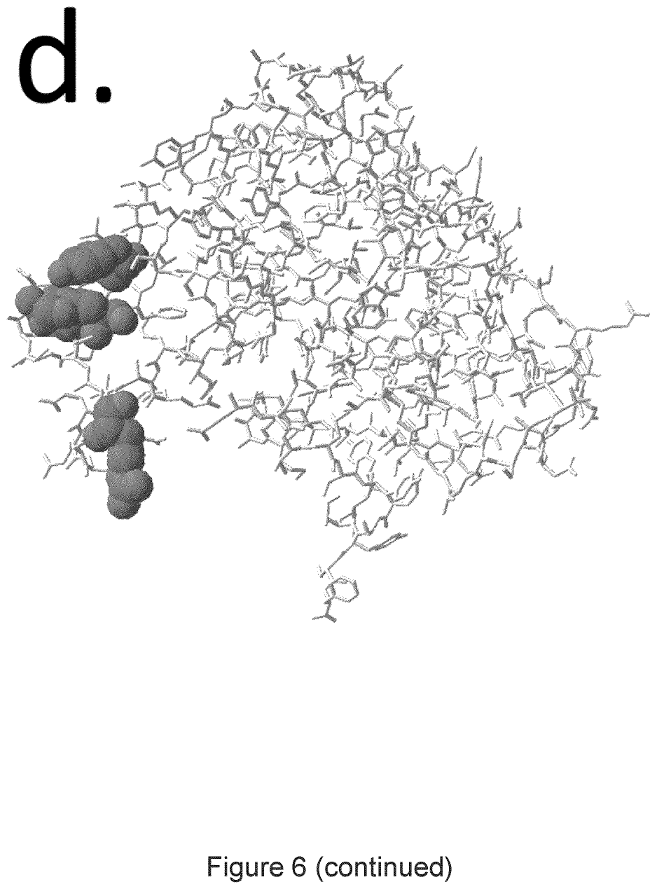

The positioning of those amino acids in the structure of the PTXa antigen are shown in FIG. 6. From a steric and/or distance perspective, some of the predicted amino acids are unlikely to be part of the epitope (i.e. considered false positives).

The results in Table 10 indicate that by normalising the 1B7 panning data with data from a panning using AbPTX-1 it is possible to clearly identify all the amino acids in the published epitope recognised by 1B7 while also removing all possible false positives indicated in the non-normalised datasets. It is also worth noting that H83 and N150, which are both in the published epitope recognised by 1B7, were ranked 13 (out of 207) and 18 (out of 207) respectively when no SA cut-off or normalisation is employed thereby underlining the value of the effect resulting from application of the AbPTX-1 normalisation.

TABLE 10
The top twelve PTXa amino acid residues bound by 1B7 according to Seqitope when a different
SA cut-off and/or normalisation is applied. Amino acid residues considered unlikely to
form part of the epitope from a steric and/or distance perspective are written in parentheses.
Amino acid residues forming part of the published epitope of 1B7 are underlined.

| | | Protocol D, | Published epitope of |
|---|---|---|---|
| Protocol D, | Protocol D, | AbPTX-1 normalisation, | 1B7 |
| no SA cut-off | SA cut-off >23 | no SA cut-off | (Sutherland et al, 2009) |
| (G22) | G78 | R79 | R79 |
| (Y89) | Y148 | E75 | H83 |
| G78 | G80 | G80 | Y148 |
| (L64) | G82 | G78 | N150 |
| Y148 | R79 | H83 | |
| (F19) | H83 | Y148 | |
| G80 | E75 | G82 | |
| G82 | N150 | T81 | |
| (F50) | (N174) | G151 | |
| R79 | (P15) | N150 | |
| (R92) | (D94) | I152 | |
| (G86) | T81 | A77 | |
| Positions considered | Positions considered | Positions considered | N/A |
| unlikely = 7 | unlikely = 3 | unlikely = 0 | |

### Example 5

Similar to Example 4, removal of false positives from the SARS-COV2-RBD dataset did not involve the use of a DYKDDDDK control panning experiment. Instead, Seqitope data from AbRBD-1 was used to normalise the Ty1 data presented in Example 2. The protocol for the normalisation is as described for the DYKDDDDK control panning normalisation carried out in Example 3.

Table 11 presents the top twelve epitope residues predicted by Seqitope for the Ty1 antibody using:
1) Protocol E, no normalisation, no SA cut-off;
2) Protocol E, no normalisation, SA cut-off of >23; and,
3) Protocol E, AbRBD-1 normalisation, no SA cut-off.

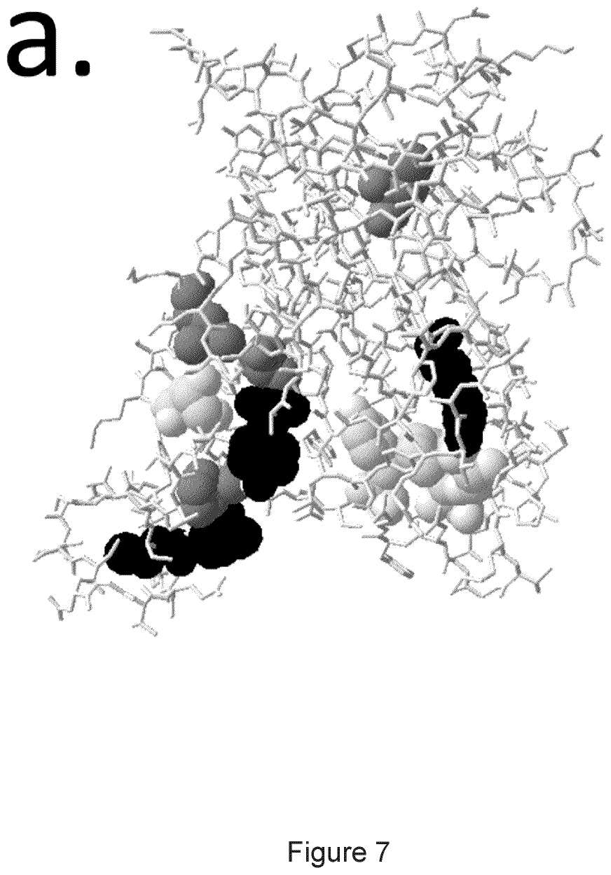

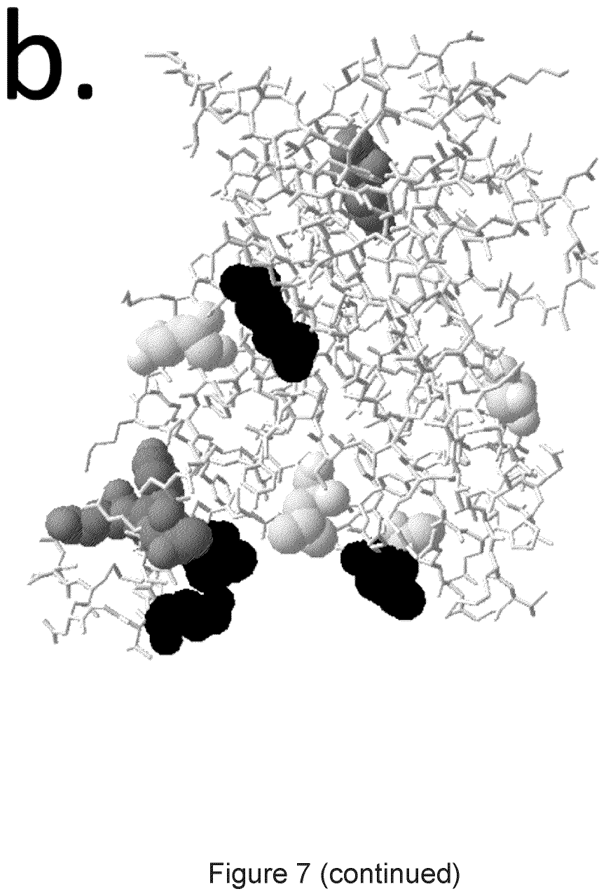

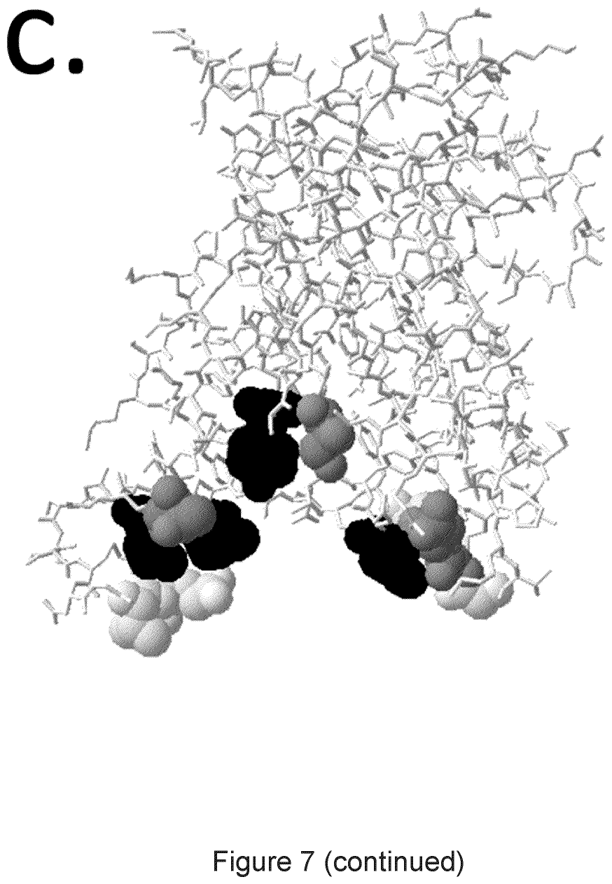

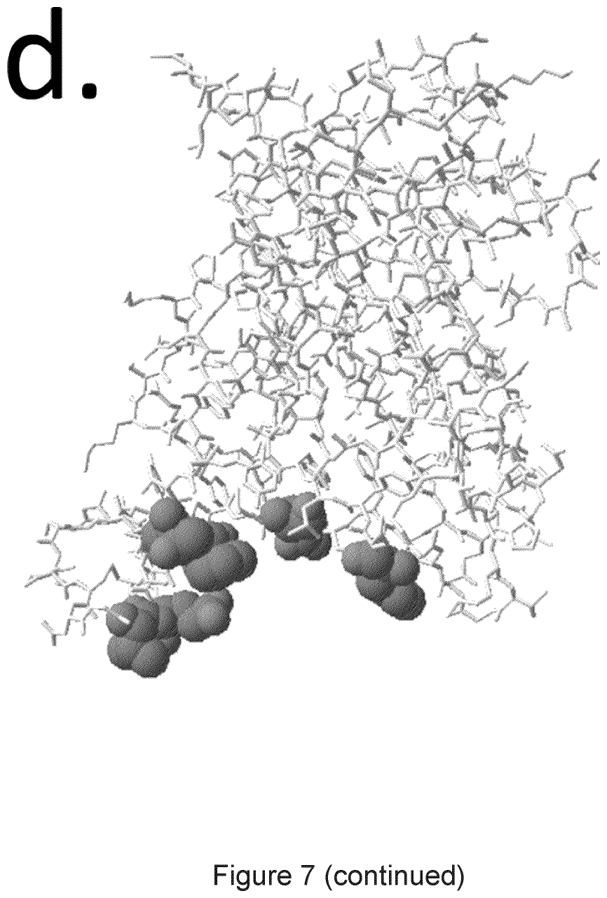

The positioning of those amino acids in the structure of the SARS-COV2-RBD antigen are shown in FIG. 7. From a steric and/or distance perspective, some of the predicted amino acids are unlikely to be part of the epitope (i.e. considered false positives).

TABLE 11
The top twelve SARS-CoV2-RBD amino acid residues bound by Ty1 according to
Seqitope when a different SA cut-off and/or normalisation is applied. Amino
acid residues considered unlikely to form part of the epitope from a steric
and/or distance perspective are written in parentheses. Amino acid residues
forming part of the published epitope of Ty1 are underlined.

| | | Protocol E, | Published epitope |
|---|---|---|---|
| Protocol E, | Protocol E, | AbRBD-1 normalisation, | of Ty1 |
| no SA cut-off | SA >23 | no SA cut-off | (Hanke et al, 2020) |
| Y351 | F490 | I472 | F490 |
| (C488) | E484 | F490 | E484 |
| (R509) | (R355) | Y449 | Y449 |
| F490 | Y449 | Y351 | T470 |
| (L461) | T470 | T470 | V483 |
| P491 | (Y380) | N448 | Q493 |
| (N422) | (Y473) | S349 | |
| (C432) | (Q474) | G447 | |
| (D467) | (N437) | E484 | |
| (F497) | G496 | V483 | |
| (D442) | (E465) | G446 | |
| (Y495) | L452 | G496 | |
| Positions considered | Positions considered | Positions considered | N/A |
| unlikely = 9 | unlikely = 6 | unlikely = 0 | |

The results in Table 11 indicate that by normalising the Ty1 panning data with data from a panning using AbRBD-1 is possible to clearly identify the five out of six the amino acids in the published epitope recognised by Ty1 (i.e. only Q493 was not identified in the top twelve), while also removing all possible false positives indicated in the non-normalised datasets. It should also be noted that when no SA cut-off or normalisation was used, E484, Y449, T4870, and V483, which are all in the published epitope recognised by Ty1, were ranked 17, 31, 39 and 74 respectively. For all protocols tested Q493 was among the bottom five ranking amino acid positions.

Seqitope ranks amino acid residues according to the ratio of the amino acid diversity. That diversity is measured using Shannon entropy by comparing a reference library and the post-panning library. Interestingly, the amino acid residues ranked the lowest often have an enrichment of non-wildtype amino acids.

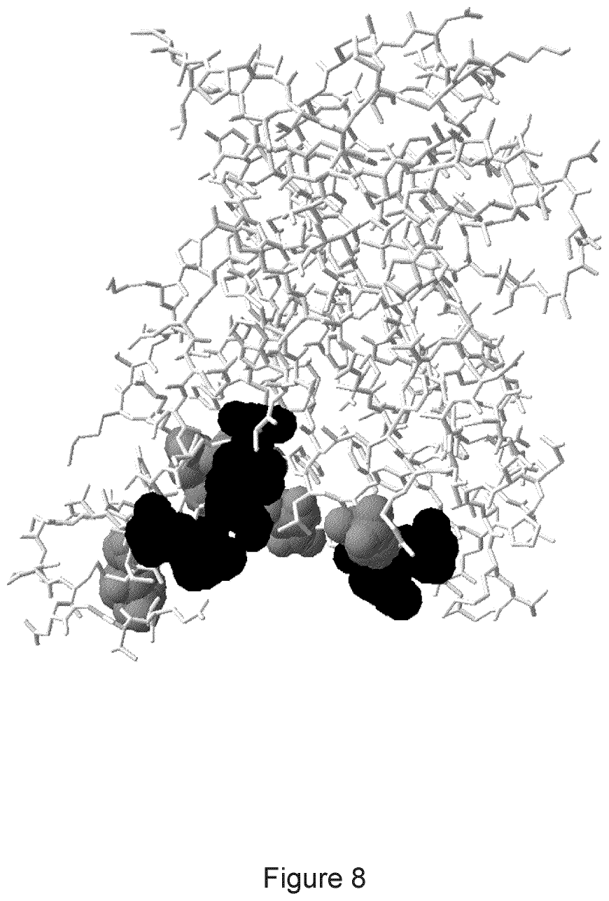

The six lowest ranked amino acid residues in the Ty1 panning after AbRBD-1 normalisation were Q493, L455, N487, F456, F486, and N450. When a 3D structural model of SARS-CoV2 is considered, all of the six lowest ranking amino acid residues are located in the same region as the six top-ranked amino acid residues (see FIG. 8). The mutation pattern of those residues is not random. Instead, very specific mutations have been enriched (see Table 12). Those specific mutations may contribute to increased binding of the antibody (i.e. an “antigen affinity maturation”).

TABLE 12
The six amino acid positions of SARS-CoV2-RBD with the lowest
Seqitope ranking when mapping Ty1. Point mutations with the
highest enrichment factor are listed for each position.

| Mutation | Enrichment Factor |
|---|---|
| Q493R | 4.2 |
| L455Q | 10.1 |
| N487D | 1.8 |
| F456S | 3.0 |
| F486I | 2.4 |
| N450Y | 11.3 |

For instance, Q493 has previously been indicated as contributing to binding of Ty1 using a proximity-based epitope mapping method (Hanke et al, 2020). Seqitope amino acid ranking is based on contribution to antibody binding and ranks Q493 very low on the list, indicating it may not be a main binding energy contributor. However, this example shows a 4.2-fold enrichment of the specific mutation Q493R which indicates that this mutation may contribute to increased Ty1 binding energy when compared to the wildtype Q493.

### Example 6

To demonstrate that the mutations listed in Table 12 are indeed increasing the affinity of the RBD-Ty1 interaction, two RBD mutants were created.
RBD-R (carrying the Q493R mutation)
RBD-YQ (carrying the N450Y and L455Q mutations)

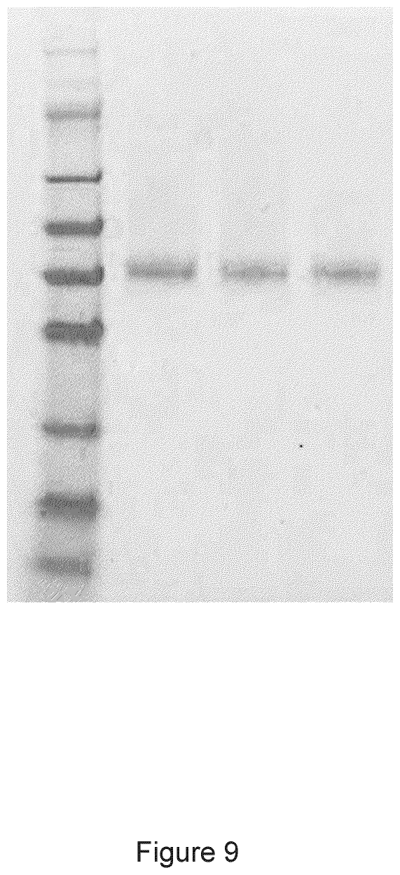

These mutants alongside the RBD wildtype (RBD-wt) sequence were produced in Expi293F cells and purified. Purity and lack of degradation of the produced proteins were assessed using SDS PAGE (see FIG. 9).

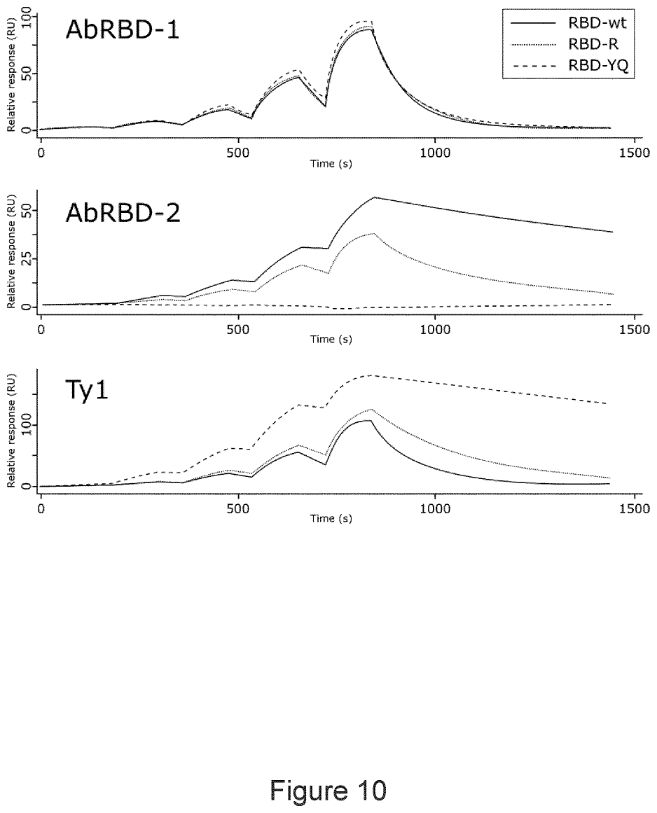

The affinity of Ty1 towards the different RBD variants were investigated using surface plasmon resonance (SPR). Besides Ty1, two other anti-RBD mAbs were used as controls. The epitope of AbRBD-1 (Y380, S383, P384, K386) is located distal from the introduced mutations i.e. this antibody should bind RBD-wt, RBD-R and RBD-YQ equally well. The epitope of AbRBD-2 (epitope Q493, N487, F456, F486) includes residue Q493 as the most important for interaction with RBD. Mutating Q493 will likely affect the binding of AbRBD-2. The affinities of Ty1, AbRBD-1 and AbRBD-2 to RBD-wt, RBD-R and RBD-YQ as analyzed using SPR are reported in Table 13. with corresponding sensorgrams in FIG. 10.

TABLE 13
ka, kd and Kd values as measured by SPR. Affinity improvement is defined
as Kd[wt]/Kd[mutant]. No binding could be detected for AbRBD-2 vs. RBD-R.

| Antibody | Antigen | ka (1/Ms) | kd (1/s) | Kd (M) | Affinity improvement |
|---|---|---|---|---|---|
| AbRBD-1 | RBD-wt | 1.02E+06 | 6.42E−02 | 6.28E−08 | (1.00) |
| | RBD-R | 4.82E+05 | 2.94E−02 | 6.10E−08 | 1.03 |
| | RBD-YQ | 8.42E+05 | 3.88E−02 | 4.61E−08 | 1.36 |
| AbRBD-2 | RBD-wt | 1.09E+05 | 6.73E−04 | 6.17E−09 | (1.00) |
| | RBD-R | — | — | — | — |
| | RBD-YQ | 5.90E+05 | 1.08E−02 | 1.83E−08 | 0.34 |
| Ty1 | RBD-wt | 8.64E+05 | 3.58E−02 | 4.15E−08 | (1.00) |
| | RBD-R | 1.77E+05 | 4.97E−03 | 2.80E−08 | 1.48 |
| | RBD-YQ | 2.23E+05 | 4.73E−04 | 2.13E−09 | 19.48 |

As expected, the control antibody AbRBD-1 bound all RBD variants with an affinity within the same range. As expected, the other control antibody AbRBD-2 had no binding to RBD-R, while the RBD-YQ had reduced affinity. Interestingly, the Ty1 had an almost 20 times increased affinity to RBD-YQ carrying the top two mutations in Table 12, confirming that Seqitope can be used for affinity maturation.

1. A method for identifying and/or characterising the target binding site of an agent or fragment thereof, the method comprising:
(i) providing an agent or fragment thereof to be tested;
(ii) providing a library of displayed polypeptides, wherein the library comprises a plurality of polypeptide molecules having different sequences derived from the target of the agent or fragment thereof;
(iii) screening the library for polypeptide molecules to which the agent or fragment thereof binds, and identifying a first population of polypeptide molecules to which the agent or fragment thereof binds and/or a second population of polypeptide molecules to which the agent or fragment thereof does not bind;
(iv) determining the sequences of the first and/or second population of polypeptide molecules;
(v) identifying and/or characterising the target binding site of the agent or fragment thereof from the information obtained in step (iv);
wherein polypeptides that are not properly displayed are excluded from the step of identifying and/or characterising the target binding site in step (v).

2. The method according to claim 1, wherein the plurality of polypeptide molecules is encoded by a plurality of polynucleotides.

3. The method according to claim 1, wherein the different sequences are derived from a reference sequence of the target of the agent or fragment thereof.

4. The method according to claim 1, wherein the target binding site is a conformational binding site or is a linear binding site.

5. The method according to claim 1, wherein one or more amino acid of the target binding site is identified and/or characterised.

6. The method according to claim 1, wherein the agent is one or more selected from the group comprising: an antibody or fragment thereof, a protein or fragment thereof, a carbohydrate or fragment thereof, a lipid or fragment thereof, a nucleic acid or fragment thereof, or a small molecule or fragment thereof, and combinations thereof.

7. The method according to claim 6, wherein the agent is an antibody or fragment thereof, and wherein the target binding site is the antibody epitope.

8. The method according to claim 1, wherein the agent is provided as part of a biological sample or is derived from a biological sample.

9. The method according to claim 8, wherein the biological sample is one or more selected from the group comprising: blood, serum, plasma, urine, saliva, cerebrospinal fluid (CSF), a virology swab, a biopsy sample, and combinations thereof.

10. The method according to claim 1, wherein the library of displayed polypeptides in step (ii) does not comprise the complete and/or full-length target of the agent or fragment thereof.

11. The method according to claim 1, wherein polypeptides that are not properly displayed are excluded from the step of identifying and/or characterising the target binding site in step (v) by:
excluding polypeptides that are not properly displayed from the library; and/or
excluding polypeptides that are not properly displayed from the first and/or second population of polypeptide molecules; and/or
excluding information about polypeptides that are not properly displayed from the information in step (iv).

12. The method according to claim 1, wherein the method comprises the further step of identifying polypeptides that are not properly displayed using an agent capable of selectively binding to properly displayed polypeptides.

13. The method according to claim 12 wherein the agent capable of selectively binding to properly displayed polypeptides comprises a control antibody to the target.

14. The method according to claim 1, wherein each displayed polypeptide in the library comprises a control peptide which is indicative of a properly displayed polypeptide.

15. The method according to claim 14, wherein the agent capable of selectively binding to properly displayed polypeptides comprises a control antibody that selectively recognises a control peptide.

16. The method according to claim 1, wherein step (ii) comprises introducing polynucleotide sequences encoding the plurality of polypeptide molecules into a host system capable of displaying the polypeptides and expressing the polypeptides in the host system.

17. The method according to claim 1, wherein step (iii) comprises:
(a) incubating the library of displayed polypeptides with the agent or fragment thereof;
(b) immobilising the agent or fragment thereof onto a solid support;
(c) washing the solid support to remove unbound displayed polypeptides to obtain the second population of polypeptide molecules; and,
(d) eluting the bound displayed polypeptides to obtain the first population of polypeptide molecules.

18. The method according to claim 1, wherein step (iii) comprises:
(a) immobilising the agent or fragment thereof onto a solid support;
(b) incubating the library of displayed polypeptides with the agent or fragment thereof;
(c) washing the solid support to remove unbound displayed polypeptides to obtain the second population of polypeptide molecules; and,
(d) eluting the bound displayed polypeptides to obtain the first population of polypeptide molecules.

19. The method according to claim 1, wherein step (iii) further comprises performing at least one control screening of the library of displayed polypeptides.

20. The method according to claim 19, wherein the at least one control screening comprises screening the library of displayed polypeptides against a second agent or fragment thereof.

21. The method according to claim 1, wherein the library of displayed polypeptides is displayed on the outer surface of one or more virus particle, on the outer surface of one or more cell, as part of a ribosome display system, as part of a DNA display system, or as part of an mRNA display system.

22. The method according to claim 21, wherein the one or more virus particle is one or more selected from the group comprising: bacteriophage, baculovirus, lentivirus, adenovirus, tobacco mosaic virus, and avian leukosis virus.

23. The method according to claim 22, wherein the bacteriophage is one or more selected from the group comprising: M13 bacteriophage, Fd bacteriophage, T4 bacteriophage, and T7 bacteriophage.

24. The method according to claim 21, wherein the one or more cell is one or more selected from the group comprising: a yeast cell, a mammalian cell, an insect cell, and a bacterial cell.

25. The method according to claim 1, wherein the plurality of polypeptide molecules is obtained by mutagenesis of the target.

26. The method according to claim 25, wherein each of the plurality of polypeptide molecules comprises between 1 to 100 amino acid changes, preferably between 1 to 50 amino acid changes, more preferably between 1 to 10 amino acid changes, or most preferably between 1 to 3 amino acid changes, compared to the target of the agent or fragment thereof.

27. The method according to claim 25, wherein mutagenesis is performed by error-prone PCR, propagation of a polynucleotide sequence encoding the target in a bacterial mutator strain, or site-directed mutagenesis.

28. The method according to claim 1, wherein the first population of polypeptide molecules is screened for polypeptide molecules to which the agent or fragment thereof binds, to thereby identify a third population of polypeptide molecules and/or a fourth population of polypeptide molecules to which the agent or fragment thereof does not bind.

29. The method according to claim 2, wherein step (iv) comprises:
(a) amplifying the plurality of polynucleotides encoding the first and/or second population of polypeptide molecules to produce a first and/or second population of amplified polynucleotides; and,
(b) performing sequencing on the first and/or second population of amplified polynucleotides.

30. The method according to claim 1, wherein step (v) comprises comparing the information obtained in step (iv) for the first population of polypeptide molecules with information obtained for at least one reference population of polypeptides.

31. The method according to claim 30, wherein the at least one reference population of polypeptides is obtained from a control screening of the library of displayed polypeptides.

32. The method of claim 30, wherein the at least one reference population of polypeptides is:
(a) the library of displayed polypeptides prior to screening; and/or,
(b) the second population of polypeptide molecules identified in step (iii); and/or,
(c) a population of polypeptides obtained by screening the library of displayed polypeptides against a second agent or fragment thereof.

33. The method according to claim 30, wherein step (v) comprises:
(a) aligning the sequences of the first population of polypeptide molecules and/or reference population of polypeptides with the reference sequence of the target of the agent or fragment thereof;
(b) counting the frequency of adenine, thymine, guanine, and cytosine residues present at each position in the polynucleotides encoding the first population polypeptides and/or reference population of polypeptides relative to the reference sequence of the target of the agent or fragment thereof, and,
(c) converting the counts obtained in sub-step (b) into counts of amino acid residues for each position in the encoded polypeptides in order to obtain frequencies of amino acids at each position in the first population polypeptides and/or reference population of polypeptides.

34. The method according to claim 33, wherein step (v) further comprises:
(d) determining the variability at each amino acid position in the target or fragment thereof for the first population polypeptides and/or reference population of polypeptides based on the amino acid frequencies obtained in sub-step (c).

35. The method according to claim 34, wherein step (v) further comprises:
(e) identifying and/or characterising the target binding site of the agent or fragment thereof based on the ratio of the variability at each amino acid position determined for the first population of polypeptides and the variability at each amino acid position determined for the reference population of polypeptides.

36. A method for identifying a target binding site of an agent or fragment thereof and/or designing a target binding site to which binding of an agent or fragment thereof is increased or decreased, the method comprising:
(i) providing an agent or fragment thereof to be tested;
(ii) providing a library of displayed polypeptides, wherein the library comprises a plurality of polypeptide molecules having different sequences derived from the target of the agent or fragment thereof;
(iii) screening the library for polypeptide molecules to which the agent or fragment thereof binds, and identifying a first population of polypeptide molecules to which the agent or fragment thereof binds and/or a second population of polypeptide molecules to which the agent or fragment thereof does not bind;
(iv) determining the sequences of the first and/or second population of polypeptide molecules;
(v) identifying a target binding site of the agent or fragment thereof and/or designing a target binding site to which binding of the agent or fragment thereof is increased or decreased, from the information obtained in step (iv);
wherein polypeptides that are not properly displayed are excluded from the step of identifying and/or designing an epitope and/or a target binding site in step (v).

37. The method according to claim 36, wherein the plurality of polypeptide molecules is encoded by a plurality of polynucleotides.

38. The method according to claim 36, wherein the different sequences are derived from a reference sequence of the target of the agent or fragment thereof.

39. A method for identifying an agent or fragment thereof and/or designing an agent or fragment thereof, to which binding of a target antigen or fragment thereof, is increased or decreased, the method comprising:
(i) providing a target antigen or fragment thereof to be tested;
(ii) providing a library of displayed agents, wherein the library comprises a plurality of agent molecules or fragments thereof having different sequences derived from the agent or fragment thereof;
iii) screening the library for agent molecules to which the antigen or fragment thereof binds, and identifying a first population of agent molecules to which the antigen or fragment thereof binds and/or a second population of agent molecules to which the antigen or fragment thereof does not bind;
(iv) determining the sequences of the first and/or second population of agent molecules;
(v) identifying and/or designing an agent or fragment thereof to which binding of the antigen or fragment thereof is increased or decreased, from the information obtained in step (iv);
wherein agent molecules that are not properly displayed are excluded from the step of identifying and/or designing an agent or fragment thereof in step (v).

40. The method according to claim 39, wherein the plurality of agent molecules is encoded by a plurality of polynucleotides.

41. The method according to claim 39, wherein the different sequences are derived from a reference sequence of the target of the agent or fragment thereof.

42. The method according to claim 39, wherein the agent or fragment thereof is an antibody or fragment thereof.

43. A library of displayed polypeptides obtained using the method according to claim 1.

44. A library of displayed agents obtained using the method according to claim 39.

45. (canceled)


SEQUENCE LISTING (25 sequences)

SEQ ID NO: 1 - protein, 8 aa, synthetic construct
DYKDDDDK

SEQ ID NO: 2 - protein, 4 aa, synthetic construct
KDIR

SEQ ID NO: 3 - DNA, 36 bp, synthetic construct
  source 1..36: mol_type = other DNA
tatactaccggtgacgatcctcccgccaccgtatac

SEQ ID NO: 4 - DNA, 37 bp, synthetic construct
  source 1..37: mol_type = other DNA
atgtatgcggccgcttcggcctgccgcgccatgcaag

SEQ ID NO: 5 - DNA, 36 bp, synthetic construct
  source 1..36: mol_type = other DNA
atacaaccggtagggtacaacctacggaatctatcg

SEQ ID NO: 6 - DNA, 37 bp, synthetic construct
  source 1..37: mol_type = other DNA
tacatgcggccgcaaaattgacgcatttattcttcac

SEQ ID NO: 7 - DNA, 33 bp, synthetic construct
  source 1..33: mol_type = other DNA
atacaaccggtagtgctaaagaacttagatgtc

SEQ ID NO: 8 - DNA, 41 bp, synthetic construct
  source 1..41: mol_type = other DNA
tacatgcggccgctgaattctcagccctcttcaaaaacttc

SEQ ID NO: 9 - DNA, 54 bp, synthetic construct
  source 1..54: mol_type = other DNA
acactctttccctacacgacgctcttccgatctaccgtggcccaggcgaccggt

SEQ ID NO: 10 - DNA, 41 bp, synthetic construct
  source 1..41: mol_type = other DNA
agacgtgtgctcttccgatctccaccaccctctgcggccgc

SEQ ID NO: 11 - DNA, 42 bp, synthetic construct
  source 1..42: mol_type = other DNA
agacgtgtgctcttccgatctgtgtgccagatatccgctctg

SEQ ID NO: 12 - DNA, 54 bp, synthetic construct
  source 1..54: mol_type = other DNA
acactctttccctacacgacgctcttccgatctcgaacgcgccggcaggggcac

SEQ ID NO: 13 - DNA, 42 bp, synthetic construct
  source 1..42: mol_type = other DNA
agacgtgtgctcttccgatctgtaaaggtagttataatttcc

SEQ ID NO: 14 - DNA, 54 bp, synthetic construct
  source 1..54: mol_type = other DNA
acactctttccctacacgacgctcttccgatctgcaaattgcgccagggcagac

SEQ ID NO: 15 - DNA, 25 bp, synthetic construct
  source 1..25: mol_type = other DNA
aatgtgagttagctcactcattagg

SEQ ID NO: 16 - DNA, 45 bp, synthetic construct
  source 1..45: mol_type = other DNA
ctttgatagctgcgggggcgaaaggtttggagtatgtctttatgc

SEQ ID NO: 17 - DNA, 44 bp, synthetic construct
  source 1..44: mol_type = other DNA
cctttcgcccccgcagctatcaaagaactgagagtgattgagag

SEQ ID NO: 18 - DNA, 41 bp, synthetic construct; identical to SEQ ID NO: 8
  source 1..41: mol_type = other DNA

SEQ ID NO: 19 - DNA, 33 bp, synthetic construct; identical to SEQ ID NO: 7
  source 1..33: mol_type = other DNA

SEQ ID NO: 20 - DNA, 36 bp, synthetic construct
  source 1..36: mol_type = other DNA
atagactcttaagtaccggtagggtacaacctacgg

SEQ ID NO: 21 - DNA, 36 bp, synthetic construct
  source 1..36: mol_type = other DNA
tacatgcggccgctttattcttcaccagattagtag

SEQ ID NO: 22 - DNA, 38 bp, synthetic construct
  source 1..38: mol_type = other DNA
gttatttcccattgcgatcctatggttttcaaccgacg

SEQ ID NO: 23 - DNA, 43 bp, synthetic construct
  source 1..43: mol_type = other DNA
gttgaaaaccataggatcgcaatgggaaataacaattgaatcc

SEQ ID NO: 24 - DNA, 49 bp, synthetic construct
  source 1..49: mol_type = other DNA
ggaaattattactacctttacaggcagttccggaagagtaatctgaagc

SEQ ID NO: 25 - DNA, 53 bp, synthetic construct
  source 1..53: mol_type = other DNA
ctcttccggaactgcctgtaaaggtagtaataatttccccctacctttgagtc